SEQUENCE LISTING (4 sequences)

SEQ ID NO: 1 - DNA, 1398 bp, Homo sapiens
atggagttggggctgtgctgggttttccttgttgctattttagaaggtgtccagtgtgaggtgcagctggtggagtctgggggaggcttggtacagcctggggggtccctgagactctcctgtgcagcctctggattcaccttcagtagttttagtatgacctgggtccgccaggctccaggaaaggggctggagtgggtttcatacattagtagtagaagtagtaccatatcctacgcagactctgtgaagggccgattcaccatctccagagacaatgccaagaactcactgtatctgcaaatgaacagcctgagagacgaggacacggctgtgtattactgtgcgagagatcctcttctagcgggagctaccttctttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - protein, 466 aa, Homo sapiens
MELGLCWVFLVAILEGVQCEVQLVESGGGLVQPGGSLRLSCAASGFTFSSFSMTWVRQAPGKGLEWVSYISSRSSTISYADSVKGRFTISRDNAKNSLYLQMNSLRDEDTAVYYCARDPLLAGATFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - DNA, 705 bp, Homo sapiens
atggaaaccccagcgcagcttctcttcctcctgctactctggctcccagataccaccggagaatttgtgttgacgcagtctccaggcaccctgtctttgtctccaggggaaagagccaccctctcctgcagggccagtcagagtgttagcagcagttacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 4 - protein, 235 aa, Homo sapiens
METPAQLLFLLLLWLPDTTGEFVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2009/0117103 A1
(43) Pub. Date: May 7, 2009

(54) M-CSF Antibody compositions Having Reduced Levels of Endotoxin

(75) Inventors: Madhav Devalaraja, Potomac, MD (US); Ronald W. Fedechko, Chesterfield, MO (US)

Correspondence Address: PHARMACIA CORPORATION;GLOBAL PATENT DEPARTMENT, POST OFFICE BOX 1027, ST. LOUIS, MO, 63006, US

(73) Assignee: Pharmacia & UpJohn Company LLC, New York, NY (US)

(21) Appl. No.: 11/817,947

(22) PCT Filed: Mar. 2, 2006
(86) PCT No.: PCT/US06/07553

§ 371 Date: Jul. 28, 2008

Related U.S. Application Data
(60) Provisional application No. 60/659,765, filed on Mar. 8, 2005.

(51) Int. Cl. A61K 39/395; C07K 1/16; A61P 37/04

(52) U.S. Cl. 424/133.1; 530/417

(57) ABSTRACT

The present invention provides for compositions of anti-M-CSF antibodies that are substantially free of endotoxin. Also provided are methods of treating M-CSF-mediated disorders with pharmaceutical formulations of anti-M-CSF antibodies having reduced endotoxin levels, including inflammatory diseases and neoplasia disorders.

### CROSS-REFERENCE TO RELATED PATENTS AND PATENT APPLICATIONS

This application claims the benefit of U.S. Patent Application Ser. No. 60/659,765, filed Mar. 8, 2005, which is incorporated by reference herein in its entirety.

### BACKGROUND OF THE INVENTION

Endotoxins are low molecular weight complexes of about 10 Kilodaltons (kDa) associated with the outer cell wall of gram-negative bacteria that can produce pyrogenic reactions upon parenteral administration to a patient. It is characterized by having an overall negative charge, high heat stability and high-molecular weight. Endotoxin is a complex of lipid, carbohydrate and protein. The lipid and carbohydrate components form a lipopolysaccharide. The lipopolysaccharide consists of three distinct chemical regions, lipid A, which is the innermost region, an intermediate core polysaccharide, and an outermost O-specific polysaccharide side chain, which is responsible for endotoxin's particular pyrogenic response.

Because of their potential pyrogenicity, endotoxin levels should be minimized and controlled in any process involving parenterally administered pharmaceuticals. Accordingly, regulatory agencies such as the United States Food & Drug Administration (FDA) has set an upper limit of 5 EU per dose per kilogram body weight in a single one-hour period for intravenous drug applications. See, e.g., The United States Pharmacopoeial Convention (USP), Pharmacopeial Forum 26 (1):223 (2000).

Included in the proteins commonly used for parentally administered pharmaceuticals are antibodies. One antibody useful for medical therapies is an antibody, which specifically binds to macrophage colony stimulating factor (M-CSF).

M-CSF is an important regulator of the function, activation, and survival of monocytes/macrophages. A number of animal models have confirmed the role of M-CSF in various diseases, including rheumatoid arthritis and cancer. Macrophages comprise key effector cells in rheumatoid arthritis. The degree of synovial macrophage infiltration in rheumatoid arthritis has been shown to closely correlate with the extent of underlying joint destruction. M-CSF, endogenously produced in the rheumatoid joint by monocytes/macrophages, fibroblasts, and endothelial cells, acts on cells of the monocyte/macrophage lineage to promote their survival and differentiation into bone destroying osteoclasts, and enhance pro-inflammatory cellular functions such as cytotoxicity, superoxide production, phagocytosis, chemotaxis and secondary cytokine production.

There is a need in the art for formulations of M-CSF antibodies that can be used to treat diseases such as rheumatoid arthritis, cancer, and other M-CSF mediated diseases, which have reduced levels of endotoxin.

### SUMMARY

In one aspect, the present invention provides compositions comprising at least one antibody comprising an amino acid sequence that is at least 90% identical to a light chain amino acid sequence shown in SEQ ID NO: 4, and further comprising an amino acid sequence that is at least 90% identical to a heavy chain amino acid sequence shown in SEQ ID NO: 2, wherein the antibody binds to human M-CSF and the composition is substantially free of endotoxin.

The present invention also provides methods of purifying a monoclonal IgG antibody comprising: contacting the antibody with an affinity chromatography resin that binds to the antibody; washing the affinity chromatography resin with a wash solution comprising phosphate ions and chloride ions; washing the affinity chromatography resin with a wash solution comprising acetate ions at pH 5.5; eluting the antibody from the affinity chromatography resin to form an affinity chromatography eluent comprising the antibody; contacting the affinity chromatography eluent with an ion-exchange resin that binds to the antibody; and eluting the antibody from the ion-exchange resin.

The present invention also provides methods of reducing the amount of endotoxin in a composition comprising at least one antibody comprising an amino acid sequence that is at least 90% identical to a light chain amino acid sequence shown in SEQ ID NO: 4, and further comprising an amino acid sequence that is at least 90% identical to a heavy chain amino acid sequence shown in SEQ ID NO: 2, wherein the antibody binds to human M-CSF, the method comprising: contacting the composition with an affinity chromatography resin that binds to the antibody; eluting the antibody from the affinity chromatography resin to form an affinity chromatography eluent comprising the antibody; contacting the affinity chromatography eluent with an ion-exchange resin that binds to the antibody; and eluting the antibody from the ion-exchange resin, wherein the antibody is substantially free of endotoxin.

The present invention also provides liquid pharmaceutical compositions comprising at least one antibody comprising an amino acid sequence that is at least 90% identical to a light chain amino acid sequence shown in SEQ ID NO: 4, and further comprising an amino acid sequence that is at least 90% identical to a heavy chain amino acid sequence shown in SEQ ID NO: 2, wherein the antibody binds to human M-CSF and the composition is substantially free of endotoxin; and a pharmaceutically acceptable excipient.

The present invention also provides methods for the treatment of a M-CSF-mediated disorder in a subject, comprising administering to the subject a therapeutically effective amount of a liquid pharmaceutical composition comprising: at least one antibody comprising an amino acid sequence that is at least 90% identical to a light chain amino acid sequence shown in SEQ ID NO: 4, and further comprising an amino acid sequence that is at least 90% identical to a heavy chain amino acid sequence shown in SEQ ID NO: 2, wherein the antibody binds to human M-CSF and the composition is substantially free of endotoxin; and a pharmaceutically acceptable excipient.

### BRIEF DESCRIPTION OF THE DRAWINGS

FIG. 1, comprising FIGS. 1A-1D, shows the nucleotide and amino acid sequences for anti-M-CSF antibody 8.11.3F. FIG. 1A shows the full length nucleotide sequence for the 8.11.3F heavy chain (SEQ ID NO: 1). FIG. 1B shows the full length amino acid sequence for the 8.11.3F heavy chain (SEQ ID NO: 2), and the amino acid sequence for the 8.11.3F heavy chain variable region is in upper case and designated between brackets “[ ]” (SEQ ID NO: 5). The amino acid sequence of each 8.11.3F heavy chain CDR is underlined and in lowercase. The heavy chain CDR amino acid sequences are as follows: CDR1: GFTFSSFSMT (SEQ ID NO: 7); CDR2: YISSRSSTISYADSVKG (SEQ ID NO: 8); and CDR3: DPLLAGATFFDY (SEQ ID NO: 9). FIG. 1C shows the nucleotide sequence for the 8.11.3F light chain (SEQ ID NO: 3). FIG. 1D shows the amino acid sequence of the full length 8.11.3F light chain (SEQ ID NO: 4), and the 8.11.3F light chain variable region is in upper case and designated between brackets “[ ]” (SEQ ID NO: 6). The amino acid sequence of each light chain CDR amino acid sequence is indicated as follows: CDR1: RASQSVSSSYLA (SEQ ID NO: 10); CDR2: GASSRAT (SEQ ID NO: 11); and CDR3: QQYGSSPLT (SEQ ID NO: 12).

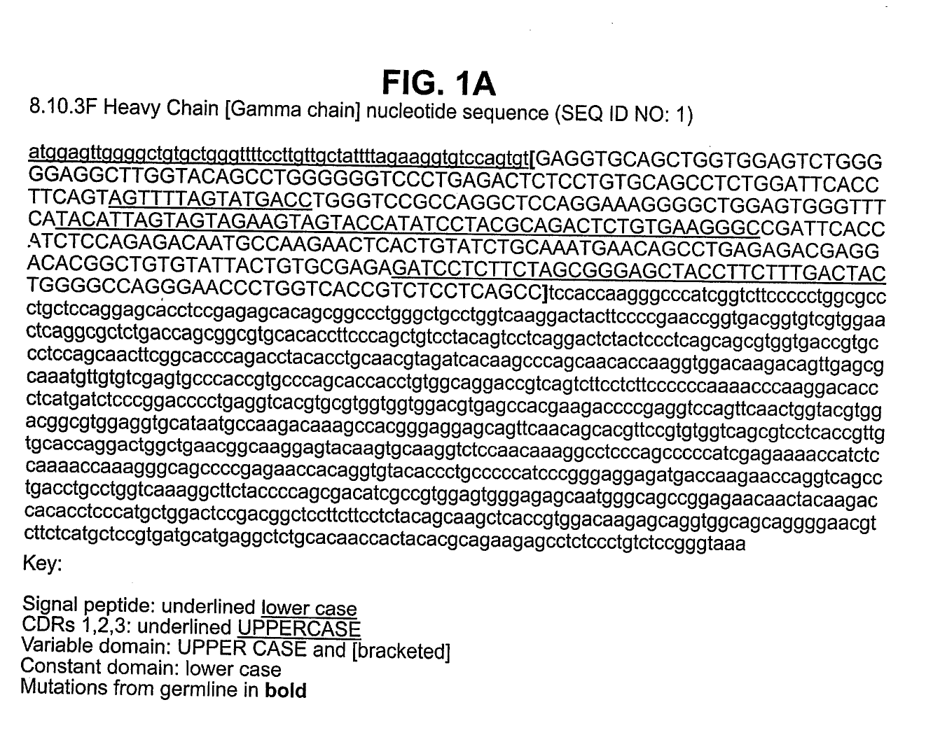

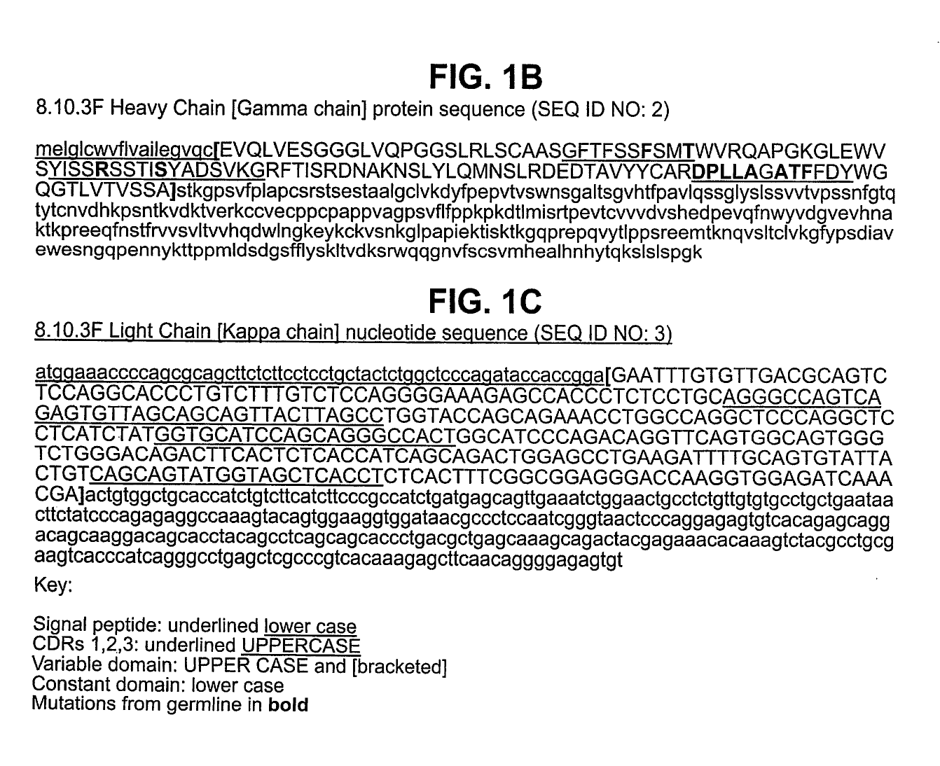

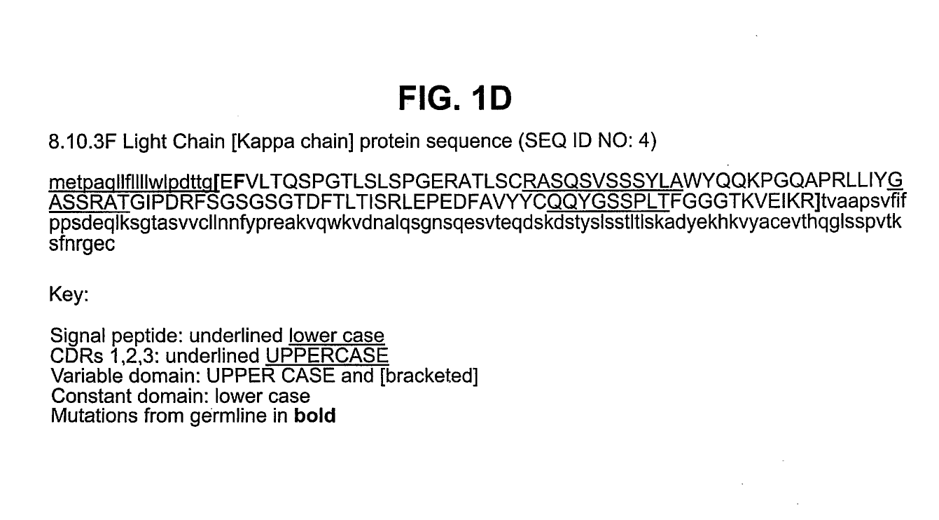

### DETAILED DESCRIPTION OF THE PREFERRED EMBODIMENTS

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990). Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of subjects.

### DEFINITIONS

In order to aid the reader in understanding the following detailed description, the following definitions are provided:

As used herein, the term “antibody” refers to an intact antibody or an antigen-binding portion that competes with the intact antibody for specific binding. See generally, Fundamental Immunology, Ch. 7 (Paul, W., ed., 2nd ed. Raven Press, N.Y. (1989). Antigen-binding portions may be produced by recombinant DNA techniques or by enzymatic or chemical cleavage of intact antibodies. In some embodiments, antigen-binding portions include Fab, Fab′, F(ab′)₂, Fd, Fv, dAb, and complementarity determining region (CDR) fragments, single-chain antibodies (scFv), chimeric antibodies, diabodies and polypeptides that contain at least a portion of an antibody that is sufficient to confer specific antigen binding to the polypeptide. From N-terminus to C-terminus, both the mature light and heavy chain variable domains comprise the regions FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md. (1987 and 1991)), Chothia & Lesk, J. Mol. Biol. 196:901-917 (1987), or Chothia et al., Nature 342:878-883 (1989).

In some embodiments, the antibody is a single-chain antibody (scFv) in which a V_{L} and V_{H} domains are paired to form a monovalent molecules via a synthetic linker that enables them to be made as a single protein chain. Bird et al., Science 242:423-426 (1988) and Huston et al., Proc. Natl. Acad. Sci. USA 85:5879-5883 (1988)). In some embodiments, the antibodies are diabodies, i.e., are bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites. See e.g., Holliger P. et al., Proc. Natl. Acad. Sci. USA 90:6444-6448 (1993) and Poljak R. J. et al., Structure 2:1121-1123 (1994). In some embodiments, one or more CDRs from an antibody of the invention may be incorporated into a molecule either covalently or noncovalently to make it an immunoadhesin that specifically binds to M-CSF. In such embodiments, the CDR(s) may be incorporated as part of a larger polypeptide chain, may be covalently linked to another polypeptide chain, or may be incorporated noncovalently.

As used herein, an antibody that is referred to by number is the same as a monoclonal antibody that is obtained from the hybridoma of the same number. For example, monoclonal antibody 8.10.3F is the same antibody as one obtained from hybridoma 8.10.3F. For example, monoclonal antibody 8.10.3F has the same heavy and light chain amino acid sequences as one obtained from hybridoma 8.10.3F. Thus, reference to antibody 8.10.3F includes an antibody, which has the heavy and light chain amino acid sequences shown in SEQ ID NOS. 2 and 4, respectively. It also includes an antibody lacking a terminal lysine on the heavy chain, as this is normally lost in a proportion of antibodies during manufacture.

As used herein, an Fd fragment means an antibody fragment that consists of the V_{H} and C_{H}1 domains; an Fv fragment consists of the V_{L} and V_{H} domains of a single arm of an antibody; and a dAb fragment (Ward et al., Nature 341:544-546 (1989)) consists of a V_{H} domain.

As used herein, the term “polypeptide” encompasses native or artificial proteins, protein fragments and polypeptide analogs of a protein sequence. A polypeptide may be monomeric or polymeric.

The terms “or an antigen-binding portion thereof” when used with the term “antibody” refers to a polypeptide that has an amino-terminal and/or carboxy-terminal deletion, but where the remaining amino acid sequence is identical to the corresponding positions in the naturally-occurring sequence. In some embodiments, the antigen-binding portion thereof may be at least 14, at least 20, at least 50, or at least 70, 80, 90, 100, 150 or at least 200 amino acids long.

As used herein, the terms “is capable of specifically binding” refers to when an antibody binds to an antigen with a dissociation constant that is ≦1 μM, preferably ≦1 nM and most preferably ≦10 pM. In certain embodiments, the K_{D} is 1 pM to 500 pM. In other embodiments, the K_{D} is between 500 pM to 1 μM. In other embodiments, the K_{D} is between 1 μM to 100 nM. In other embodiments, the K_{D} is between 100 mM to 10 n M.

As used herein, the term “monoclonal antibody” refers to an antibody obtained from a population of substantially homogeneous antibodies, i.e., the individual antibodies comprising the population are identical except for possible naturally occurring mutations that may be present in minor amounts or lacking a C-terminal lysine. Monoclonal antibodies are highly specific, being directed against a single antigenic site. Furthermore, in contrast to conventional (polyclonal) antibody preparations, which typically include different antibodies, directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler, et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson, et al., Nature 352:624-628 (1991) and Marks, et al., J. Mol. Biol. 222:581-597 (1991), for example.

The term “isolated protein”, “isolated polypeptide” or “isolated antibodies” is a protein, polypeptide or antibody that by virtue of its origin or source of derivation has one to four of the following: (1) is not associated with naturally associated components that accompany it in its native state, (2) is free of other proteins from the same species, (3) is expressed by a cell from a different species, or (4) does not occur in nature. Thus, a polypeptide that is chemically synthesized or synthesized in a cellular system different from the cell from which it naturally originates will be “isolated” from its naturally associated components. An isolated protein/antibody may also be rendered substantially free of naturally associated cellular components by isolation, using protein purification techniques well known in the art.

Examples of isolated/purified antibodies include an anti-M-CSF antibody that has been affinity purified using M-CSF, an anti-M-CSF antibody that has been synthesized by a hybridoma or other cell line in vitro, and a human anti-M-CSF antibody derived from a transgenic mouse. Thus, in preferred embodiments, the anti-M-CSF antibodies have a purity of at least about 95% (w/w—weight anti-M-CSF antibodies/weight of components other than pharmaceutically acceptable excipients), and in further embodiments, the anti-M-CSF antibodies have a purity from about 95% w/w to about 99.5% w/w.

An antibody is “substantially pure,” “substantially homogeneous,” or “substantially purified” when at least about 60 to 75% of a sample exhibits a single species of antibody. The antibody may be monomeric or multimeric. A substantially pure antibody can typically comprise about 50%, 60%, 70%, 80% or 90% w/w of an antibody sample, more usually about 95%, and preferably will be over 99% pure. Antibody purity or homogeneity may be indicated by a number of means well known in the art, such as polyacrylamide gel electrophoresis of an antibody sample, followed by visualizing a single polypeptide band upon staining the gel with a stain well known in the art. For certain purposes, higher resolution may be achieved by using HPLC or other means well known in the art for purification.

As used herein, the term “human antibody” is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

As used herein, the term “recombinant human antibody” is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “epitope” includes any protein determinant capable of specific binding to an immunoglobulin or T-cell receptor or otherwise interacting with a molecule. Epitopic determinants generally consist of chemically active surface groupings of molecules such as amino acids or sugar side chains and generally have specific three-dimensional structural characteristics, as well as specific charge characteristics. An epitope may be “linear” or “conformational.” In a linear epitope, all of the points of interaction between the protein and the interacting molecule (such as an antibody) occur linearly along the primary amino acid sequence of the protein. In a conformational epitope, the points of interaction occur across amino acid residues on the protein that are separated from one another.

As used herein, the term “polynucleotide” or “nucleic acid”, used interchangeably herein, means a polymeric form of nucleotides of at least 10 bases in length, either ribonucleotides or deoxynucleotides or a modified form of either type of nucleotide. The term includes single and double stranded forms.

A reference to a “polynucleotide” or a “nucleic acid” sequence encompasses its complement unless otherwise specified. Thus, a reference to a nucleic acid having a particular sequence should be understood to encompass its complementary strand, with its complementary sequence.

As used herein, the term “isolated polynucleotide” or “isolated nucleic acid” means a polynucleotide of genomic, cDNA, or synthetic origin or some combination thereof, which by virtue of its origin or source of derivation, the isolated polynucleotide has one to three of the following: (1) is not associated with all or a portion of a polynucleotide with which the “isolated polynucleotide” is found in nature, (2) is operably linked to a polynucleotide to which it is not linked in nature, or (3) does not occur in nature as part of a larger sequence.

The term “oligonucleotide” as used herein includes naturally occurring, and modified nucleotides linked together by naturally occurring and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for primers and probes; although oligonucleotides may be double stranded, e.g. for use in the construction of a gene mutant. Oligonucleotides of the invention can be either sense or antisense oligonucleotides.

As used herein, the term “naturally occurring nucleotides” includes deoxyribonucleotides and ribonucleotides. The term “modified nucleotides” as used herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al., Nucl. Acids Res. 14:9081 (1986); Stec et al., J. Am. Chem. Soc. 106:6077 (1984); Stein et al., Nucl. Acids Res. 16:3209 (1988); Zon et al., Anti-Cancer Drug Design 6:539 (1991); Zon et al., Oligonucleotides and Analogues: A Practical Approach, pp. 87-108 (F. Eckstein, Ed., Oxford University Press, Oxford England (1991)); U.S. Pat. No. 5,151,510; Uhlmann and Peyman, Chemical Reviews 90:543 (1990), the disclosures of which are hereby incorporated by reference. An oligonucleotide can include a label for detection, if desired.

As used herein, the terms “selectively hybridize” mean to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. “High stringency” or “highly stringent” conditions can be used to achieve selective hybridization conditions as known in the art and discussed herein. One example of “high stringency” or “highly stringent” conditions is the incubation of a polynucleotide with another polynucleotide, wherein one polynucleotide may be affixed to a solid surface such as a membrane, in a hybridization buffer of 6×SSPE or SSC, 50% formamide, 5×Denhardt's reagent, 0.5% SDS, 100 μg/ml denatured, fragmented salmon sperm DNA at a hybridization temperature of 42° C. for 12-16 hours, followed by twice washing at 55° C. using a wash buffer of 1×SSC, 0.5% SDS. See also Sambrook et al., supra, pp. 9.50-9.55.

As applied to polynucleotides, the terms “substantial identity”, “percent identity” or “% identical” mean the percent of residues when a first contiguous sequence is compared and aligned for maximum correspondence to a second contiguous sequence. The length of sequence identity comparison may be over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. The terms “substantial identity”, “percent identity” or “% identical” mean that when a polynucleotide molecule is optimally aligned with appropriate nucleotide insertions or deletions with another polynucleotide molecule (or its complementary strand), there is nucleotide sequence identity of at least about 85%, preferably at least about 90%, and more preferably at least about 95%, 96%, 97%, 98% or 99% of the nucleotide bases, as measured by any well-known algorithm of sequence identity, such as FASTA, BLAST or Gap. There are a number of different algorithms known in the art that can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183:63-98 (1990); Pearson, Methods Mol. Biol. 132:185-219 (2000); Pearson, Methods Enzymol. 266:227-258 (1996); Pearson, J. Mol. Biol. 276:71-84 (1998)). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

As applied to polypeptides, the terms “substantial identity”, “percent identity” or “% identical” mean that two peptide sequences, when optimally aligned, such as by the programs GAP or BESTFIT using default gap weights, as supplied with the programs, share at least 70%, 75% or 80% sequence identity, preferably at least 90% or 95% sequence identity, and more preferably at least 97%, 98% or 99% sequence identity. In certain embodiments, residue positions that are not identical differ by conservative amino acid substitutions. A “conservative amino acid substitution” is one in which an amino acid residue is substituted by another amino acid residue having a side chain R group with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity may be adjusted upwards to correct for the conservative nature of the substitution. Means for making this adjustment are well-known to those of skill in the art. See, e.g., Pearson, Methods Mol. Biol. 243:307-31 (1994). Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups are: valine-leucine-isoleucine, phenylalanine-tyrosine, lysine-arginine, alanine-valine, glutamate-aspartate, and asparagine-glutamine. Sequence identity for polypeptides, is typically measured using sequence analysis software. Protein analysis software matches sequences using measures of similarity assigned to various substitutions, deletions and other modifications, including conservative amino acid substitutions. For instance, GCG contains programs such as “Gap” and “Besffit” which can be used with default parameters, as specified with the programs, to determine sequence homology or sequence identity between closely related polypeptides, such as homologous polypeptides from different species of organisms or between a wild type protein and a mutant thereof. See, e.g., GCG Version 6.1. Polypeptide sequences also can be compared using FASTA using default or recommended parameters, see GCG Version 6.1. (University of Wisconsin Wis.) FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). Another preferred algorithm when comparing a sequence of the invention to a database containing a large number of sequences from different organisms is the computer program BLAST, especially blastp or tblastn, using default parameters, as supplied with the programs. See, e.g., Altschul et al., J. Mol. Bio. 215:403-410 (1990); Altschul et al., Nucleic Acids Res. 25:3389-402 (1997). The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences.

“Operably linked” sequences include both expression control sequences that are contiguous with the gene of interest and expression control sequences that act in trans or at a distance to control the gene of interest. The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

As used herein, the term “vector” means a nucleic acid molecule capable of transporting another nucleic acid to which it has been linked. In some embodiments, the vector is a plasmid, i.e., a circular double stranded DNA loop into which additional DNA segments may be ligated. In some embodiments, the vector is a viral vector, wherein additional DNA segments may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”).

As used herein, the terms “recombinant host cell” (or simply “host cell”) means a cell into which a recombinant expression vector has been introduced. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

A “therapeutically effective amount” refers to an amount effective, at dosages and for periods of time necessary, to achieve the desired therapeutic result, which includes treatment or prophylactic prevention of any M-CSF meditated condition, including inflammatory diseases and neoplasia disorders. It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Likewise, a therapeutically effective amount of the antibody or antibody portion may vary according to factors such as the disease state, age, sex, and weight of the individual, the ability of the antibody or antibody portion to elicit a desired response in the individual, and the desired route of administration of the antibody formulation. A therapeutically effective amount is also one in which any toxic or detrimental effects of the antibody or antibody portion are outweighed by the therapeutically beneficial effects.

As used herein, the term “subject” for purposes of treatment includes any subject, and preferably is a subject who is in need of the treatment of an M-CSF-mediated disorder. For purposes of prevention, the subject is any subject, and preferably is a subject that is at risk for, or is predisposed to, developing an M-CSF-mediated disorder. The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the invention, the subject is a human.

As used herein, the term “M-CSF-mediated disorder” is intended to include diseases and other disorders in which the presence of M-CSF in a subject suffering from the disorder is elevated in comparison to a normal healthy subject, whether the elevated M-CSF levels are now known or later evidenced or suspected of being either responsible for the pathophysiology of the disorder or a factor that contributes to a worsening of the disorder. Such disorders may be evidenced, for example, by an increase in the levels of M-CSF secreted and/or on the cell surface or increased tyrosine autophosphorylation of c-fms in the affected cells or tissues of a subject suffering from the disorder. The increase in M-CSF levels may be detected, for example, using an anti-M-CSF antibody as would be understood by one of skill in the art. Examples of M-CSF-mediated disorders that are encompassed by the present invention include inflammatory diseases, cardiovascular disorders, and neoplasia disorders.

As used herein, the terms “neoplasia” and “neoplasia disorders”, used interchangeably herein, refer to new cell growth that results from a loss of responsiveness to normal growth controls, e.g. to “neoplastic” cell growth. Neoplasia is also used interchangeably herein with the term “cancer” and for purposes of the present invention; cancer is one subtype of neoplasia. As used herein, the term “neoplasia disorder” also encompasses other cellular abnormalities, such as hyperplasia, metaplasia and dysplasia. The terms neoplasia, metaplasia, dysplasia and hyperplasia can be used interchangeably herein and refer generally to cells experiencing abnormal cell growth.

As used herein, the term “treatment” refers to both therapeutic treatment and prophylactic or preventative measures, wherein the object is to prevent or slow down (lessen) the targeted pathologic condition or condition. Those in need of treatment include those already with the condition as well as those prone to have the condition or those in whom the condition is to be prevented.

When introducing elements of the present invention or the preferred embodiment(s) thereof, the articles “a”, “an”, “the” and “said” are intended to mean that there are one or more of the elements. Throughout this specification and claims, the terms “comprising”, “comprise”, “comprises”, “including” and “having” are intended to be inclusive and mean that there may be additional elements other than the listed elements.

### Anti-M-CSF Antibodies:

In accordance with the present invention, it has been discovered that compositions can be prepared having at least one antibody comprising an amino acid sequence that is at least 90% identical to a light chain sequence shown in SEQ ID NO: 4, and further comprising an amino acid sequence that is at least 90% identical to a heavy chain amino acid sequence shown in SEQ ID NO: 2, wherein the antibody binds to human M-CSF and the composition is substantially free of endotoxin.

While not wishing to be bound by theory, it is believed that because endotoxin can cause biological effects that are of an inflammatory/pyrogenic nature, the efficacy of an endotoxin-contaminated anti-M-CSF pharmaceutical therapy such as the one described herein, which, in one embodiment, is intended to treat inflammatory M-CSF-mediated disorders, may be hindered or masked if the pharmaceutical therapy is not substantially free of endotoxin.

The present invention provides novel formulations for anti-M-CSF antibodies. As used herein, the phrase “anti-M-CSF antibody” refers to any antibody, or any portion thereof, that is capable of binding to any portion of a macrophage colony-stimulating factor (“M-CSF”) polypeptide that may be present within or isolated from any animal. In certain embodiments, the M-CSF polypeptide is a human M-CSF polypeptide.

Suitable anti-M-CSF antibodies for use with the present invention may be chosen from polyclonal or monoclonal antibodies. In certain aspects, the monoclonal anti-M-CSF antibody can be a murine, chimeric, humanized or human antibody. In further embodiments, the monoclonal anti-M-CSF antibody is a human monoclonal anti-M-CSF antibody. In further embodiments, the monoclonal anti-M-CSF antibody is an isolated monoclonal anti-M-CSF antibody. In further embodiments, the monoclonal anti-M-CSF antibody is a recombinant monoclonal anti-M-CSF antibody.

In certain embodiments, the anti-M-CSF antibodies which are suitable for use with the present invention include those anti-M-CSF antibodies and methods to prepare them that are described in U.S. Published Application No. 20050059113 to Bedian, et al. In other embodiments, the anti-M-CSF antibodies which are suitable for use with the present invention include any one or more of those anti-M-CSF monoclonal antibodies having the heavy and light chain amino acid sequences of the antibodies designated 252, 88, 100, 3.8.3, 2.7.3, 1.120.1, 9.14.41, 9.7.2IF, 9.14.4, 8.10.3, 9.7.2, 9.7.2C-Ser, 9.14.4C-Ser, 8.10.3C-Ser, 8.10.3-CG2, 9.7.2-CG2, 9.7.2-CG4, 9.14.4-CG2, 9.14.4-CG4, 9.14.4-Ser, 9.7.2-Ser, 8.10.3-Ser, 8.10.3-CG4, 8.10.3FG1 or 9.14.4G1 in U.S. Published Application No. 20050059113 to Bedian, et al. In still other embodiments, the anti-M-CSF antibodies which are suitable for use with the present invention include those anti-M-CSF monoclonal antibodies having the heavy and light chain amino acid sequences of the antibody designated 8.10.3F in U.S. Published Application No. 20050059113 to Bedian, et al.

In addition, such anti-M-CSF antibodies may be chosen based on differences in the amino acid sequences in the constant region of their heavy chains. For example, the anti-M-CSF antibodies may be chosen from the IgG class, which have “gamma” type heavy chains. The class and subclass of anti-M-CSF antibodies may be determined by any method known in the art. In general, the class and subclass of an antibody may be determined using antibodies that are specific for a particular class and subclass of antibody. Such antibodies are commercially available. The class and subclass can be determined by ELISA, or Western Blot as well as other techniques. Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

The anti-M-CSF antibody can be an IgG, an IgM, an IgE, an IgA, or an IgD molecule. In further embodiments, the anti-M-CSF antibody is an IgG and is an IgG1, IgG2, IgG3 or IgG4 subclass. One of the major mechanisms through which antibodies kill cells is through fixation of complement and participation in CDC. The constant region of an antibody plays an important role in connection with an antibody's ability to fix complement and participate in CDC. Thus, generally one selects the isotype of an antibody to either provide the ability of complement fixation, or not. In the case of the present invention, generally, as mentioned above, it is generally not preferred to utilize an antibody that kills the cells. There are a number of isotypes of antibodies that are capable of complement fixation and CDC, including, without limitation, the following: murine IgM, murine IgG2a, murine IgG2b, murine IgG3, human IgM, human IgG1, and human IgG3. In contrast, preferred isotypes which are not capable of complement fixation and CDC include, without limitation, human IgG2 and human IgG4. In addition to heavy chain sequence differences, the IgG antibodies differ within their subclass based on the number of disulfide bonds and length of the hinge region. For example, the IgG2 subclass has several differences distinct from the other subclasses. The IgG2 and IgG4 subclasses are known to have 4 disulfide bonds within their hinge region, while IgG1 has 2 and IgG3 has 11 disulfide bonds. Other differences for IgG2 antibodies include their reduced ability to cross the placenta and the inability of IgG2 antibodies to bind to lymphocyte Fc receptors. Thus, in certain embodiments, the anti-M-CSF antibody is subclass IgG2 or IgG4. In another preferred embodiment, the anti-M-CSF antibody is subclass IgG2.

In other embodiments, suitable anti-M-CSF antibodies may be chosen based on differences in the amino acid sequences in their heavy chains. For example, the anti-M-CSF antibodies of the present invention may have human gamma type heavy chains that utilize any of the following human V_{H} germline genes: V_{H}1, V_{H}2, V_{H}3, V_{H}4, or V_{H}5. For purposes of the present invention, the phrase “heavy chain variable region” is often abbreviated with the term (V_{H}). In certain embodiments, the anti-M-CSF antibodies utilize the human V_{H}3 germline gene. In further embodiments, the anti-M-CSF antibodies utilize the human V_{H} 3-48 germline gene. In still further embodiments, the anti-M-CSF antibodies utilize the D1-26 human D_{H} gene. In still further embodiments, the anti-M-CSF antibodies utilize the J_{H}4 human J_{H} gene.

In further embodiments, the anti-M-CSF antibodies may be chosen based on differences in the amino acid sequences of their light chains. For example, suitable anti-M-CSF antibodies may have lambda light chains or kappa light chains. However, in certain embodiments, the anti-M-CSF antibodies of the present invention have kappa light chains. In some embodiments, where the anti-M-CSF antibody comprises a kappa light chain, the polynucleotide encoding the variable domain of the light chain comprises a human V_{K} L5, O12, L2, B3, L15, or A27 gene and a human JK1, JK2, JK3, JK4, or JK5 gene. In some embodiments where the antibody comprises a kappa light chain, the light chain variable region (V_{L}) is encoded in part by a human V_{K}A27 gene and a human J_{K}4 gene. In particular embodiments of the invention, the light chain variable domain is encoded by human V_{K}A27/JK3 genes.

Table 1 lists the heavy chain and light chain human germline gene derivation and sequences for the anti-M-CSF monoclonal antibody 8.10.3F

TABLE 1
Heavy and Light Chain Human Gene Utilization and Sequences

| | Heavy Chain | | | | Light Chain | | |
|---|---|---|---|---|---|---|---|
| | SEQ ID | | | | SEQ ID | | |
| Antibody | NO: | V_{H} | D_{H} | J_{H} | NO: | V_{K} | J_{K} |
| 8.10.3F | 1 (nucleic | 3-48 | 1-26 | 4b | 3 (nucleic | A27 | 4 |
| | acid) | | | | acid) | | |
| | 2 (amino | | | | 4 (amino | | |
| | acid) | | | | acid) | | |

Some anti-M-CSF antibodies in accordance with the present invention were generated with a bias towards the utilization of the human V_{H} 3-48 heavy chain variable region. In XenoMouse™ mice, there are more than 30 distinct functional heavy chain variable genes with which to generate antibodies. Bias, therefore, is indicative of a preferred binding motif of the antibody-antigen interaction with respect to the combined properties of binding to the antigen and functional activity.

In some embodiments, the nucleic acid molecule encodes an amino acid sequence comprising 1, 2, 3, 4, 5, 6, 7, 8, 9, 10, 11, 12, 13, 14, 15, 16, 17 or 18 mutations compared to the germline amino acid sequence of the human V, D or J genes. In some embodiments, said mutations are in the heavy chain variable region. In some embodiments, said mutations are in the CDR regions.

In some embodiments, the nucleic acid molecule encodes one or more amino acid mutations compared to the germline sequence that are identical to amino acid mutations found in the V_{H} of monoclonal antibody 8.10.3F. In some embodiments, the nucleic acid encodes at least three amino acid mutations compared to the germline sequences that are identical to at least three amino acid mutations found in one of the above-listed monoclonal antibodies.

In some embodiments, the nucleic acid molecule encodes a V_{L} amino acid sequence comprising one or more variants compared to germline sequence that are identical to the variations found in the V_{L} of one of the antibodies 8.10.3F.

In some embodiments, the nucleic acid molecule encodes at least three amino acid mutations compared to the germline sequence found in the V_{L} of the antibody 8.10.3.

In some embodiments, the antibody is a single-chain antibody (scFvy in which a V_{L} and V_{H} domains are paired to form a monovalent molecules via a synthetic linker that enables them to be made as a single protein chain. Bird et al., Science 242:423-426 (1988) and Huston et al., Proc. Natl. Acad. Sci. USA 85:5879-5883 (1988). In some embodiments, the antibodies are diabodies, i.e., are bivalent antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites. See e.g., Holliger P. et al., Proc. Natl. Acad. Sci. USA 90:6444-6448 (1993 and Poijak R. J. et al., Structure 2:1121-1123 (1994). In some embodiments, one or more CDRs from an antibody of the invention may be incorporated into a molecule either covalently or noncovalently to make it an immunoadhesin that specifically binds to M-CSF. In such embodiments, the CDR(s) may be incorporated as part of a larger polypeptide chain, may be covalently linked to another polypeptide chain, or may be incorporated noncovalently.

In another embodiment, the anti-M-CSF antibody has selectivity (or specificity) for M-CSF that is at least 100 times greater than its selectivity for any other polypeptide. In some embodiments, the anti-M-CSF antibody does not exhibit any appreciable specific binding to any other protein other than M-CSF. One can determine the selectivity of the anti-M-CSF antibody for M-CSF using methods well known in the art following the teachings of the specification. For instance, one can determine the selectivity using Western blot, FACS, ELISA, or RIA. Thus, in some embodiments, the monoclonal anti-M-CSF antibody is capable of specifically binding to M-CSF.

In some embodiments, the C-terminal lysine of the heavy chain of the anti-M-CSF antibody of the invention is not present.

Table 1 lists the sequence identifiers (SEQ ID NOS) of the nucleic acids that comprise the heavy and light chains and the corresponding predicted amino acid sequences for the anti-M-CSF monoclonal antibody 8.10.3F. While DNA sequences encoding a signal polypeptide are shown in the sequence identifiers, the antibody typically does not comprise a signal polypeptide because the signal polypeptide is generally eliminated during post-translational modifications. In various embodiments of the invention, one or both of the heavy and light chains of the anti-M-CSF antibodies includes a signal sequence (or a portion of the signal sequence). In other embodiments of the invention, neither the heavy nor light chain of the anti-M-CSF antibodies includes a signal sequence.

In some embodiments, the nucleic acid molecule encodes a light chain amino acid sequence that is at least 70%, 75%, 80%, 85%, 90%, 95%, 97%, 98%, 99% or 100% identical to a light chain amino acid sequence of antibody 8.10.3F of SEQ ID NO: 4, or to a V_{L} amino acid sequence of SEQ ID NO 6. Nucleic acid molecules of the invention include nucleic acids that hybridize under highly stringent conditions, such as those described above, to a nucleic acid sequence encoding the light chain amino acid sequence of SEQ ID NO: 4, or that has the polynucleotide sequence of SEQ ID NO: 3.

In some embodiments, the nucleic acid molecule comprises a polynucleotide sequence that encodes the light chain amino acid sequence of monoclonal antibody 8.10.3F, or a portion thereof. In some embodiments, the nucleic acid molecule comprises a polynucleotide sequence that encodes the light chain polynucleotide sequence of monoclonal antibody 8.10.3F of SEQ ID NO: 3, or a portion thereof. In some embodiments, the nucleic acid molecule comprises a polynucleotide sequence that encodes the V_{L} amino acid sequence of monoclonal antibody 8.10.3F of SEQ ID NO: 6, or a portion thereof. In some embodiments, said portion comprises at least the CDR2 region. In some embodiments, the nucleic acid encodes the amino acid sequence of the light chain CDRs of said antibody. In some embodiments, said portion is a contiguous portion comprising CDR1-CDR3.

In some embodiments, the nucleic acid molecule encodes a heavy chain amino acid sequence that is at least 70%, 75%, 80%, 85%, 90%, 95%, 97%, 98%, 99% or 100% identical to a heavy chain amino acid sequence of antibody 8.10.3F of SEQ ID NO: 2, or to a V_{H} amino acid sequence of SEQ ID NO 5. Nucleic acid molecules of the invention include nucleic acids that hybridize under highly stringent conditions, such as those described above, to a nucleic acid sequence encoding the heavy chain amino acid sequence of SEQ ID NO: 2, or that has the polynucleotide sequence of SEQ ID NO: 1.

In some embodiments, the nucleic acid molecule comprises a polynucleotide sequence that encodes the heavy chain amino acid sequence of monoclonal antibody 8.10.3F, or a portion thereof. In some embodiments, the nucleic acid molecule comprises a polynucleotide sequence that encodes the heavy chain polynucleotide sequence of monoclonal antibody 8.10.3F of SEQ ID NO: 2, or a portion thereof. In some embodiments, the nucleic acid molecule comprises a polynucleotide sequence that encodes the V_{H} amino acid sequence of monoclonal antibody 8.10.3F of SEQ ID NO: 5, or a portion thereof. In some embodiments, said portion comprises at least the CDR2 region. In some embodiments, the nucleic acid encodes the amino acid sequence of the light chain CDRs of said antibody. In some embodiments, said portion is a contiguous portion comprising CDR1-CDR3.

In further embodiments, the nucleic acid molecule comprises a polynucleotide sequence that encodes at least a portion of the V_{H} amino acid sequence of 8.10.3F (SEQ ID NO: 5) or said sequence having conservative amino acid mutations and/or a total of three or fewer non-conservative amino acid substitutions. In various embodiments the sequence encodes one or more CDR regions, preferably a CDR3 region, all three CDR regions, a contiguous portion including CDR1-CDR3, or the entire V_{H} region.

In still further embodiments, the nucleic acid molecule comprises a polynucleotide sequence that encodes the heavy chain amino acid sequence of SEQ ID NO: 1 or a portion thereof. In still further embodiments, the nucleic acid molecule comprises a polynucleotide sequence that encodes the heavy chain variable domain amino acid sequence of SEQ ID NO: 5 or a portion thereof.

In another embodiment, the nucleic acid encodes a full-length light chain of an antibody selected from 8.10.3F, or a light chain comprising the amino acid sequence of SEQ ID NO: 4 and a constant region of a light chain, or a light chain comprising a mutation. Further, the nucleic acid may comprise the light chain polynucleotide sequence of SEQ ID NO: 3 and the polynucleotide sequence encoding a constant region of a light chain, or a nucleic acid molecule encoding a light chain comprise a mutation.

In some embodiments, the nucleic acid molecule comprises a polynucleotide sequence that encodes at least a portion of the V_{H} amino acid sequence of 8.10.3F (SEQ ID NO: 5) or said sequence having conservative amino acid mutations and/or a total of three or fewer non-conservative amino acid substitutions. In various embodiments the sequence encodes one or more CDR regions, preferably a CDR3 region, all three CDR regions, a contiguous portion including CDR1-CDR3, or the entire V_{H} region.

In another aspect of the invention, the anti-M-CSF antibodies demonstrate both species and molecule selectivity. In some embodiments, the anti-M-CSF antibody binds to human, cynomologus monkey and mouse M-CSF. Following the teachings of the specification, one may determine the species selectivity for the anti-M-CSF antibody using methods well known in the art. For instance, one may determine the species selectivity using Western blot, FACS, ELISA, RIA, a cell proliferation assay, or an M-CSF receptor-binding assay. In a preferred embodiment, one may determine the species selectivity using a cell proliferation assay or ELISA. In another embodiment, the anti-M-CSF antibody has selectivity for M-CSF that is at least 100 times greater than its selectivity for GM-/G-CSF. In some embodiments, the anti-M-CSF antibody does not exhibit any appreciable specific binding to any other protein other than M-CSF. One can determine the selectivity of the anti-M-CSF antibody for M-CSF using methods well known in the art following the teachings of the specification. For instance one can determine the selectivity using Western blot, FACS, ELISA, or RIA.

### Endotoxin

The adverse health effects of endotoxin are related to the amount of endotoxin in the product dose administered to a subject. Because the dose may vary from product to product, the endotoxin limit is expressed as K/M. K is 5.0 EU/kilogram (kg), which represents the approximate threshold pyrogen dose for humans and rabbits. That is the level at which a product is adjudged pyrogenic or non-pyrogenic. M represents the rabbit pyrogen test dose or the maximum human dose per kilogram that would be administered in a single one-hour period, whichever is larger. The FDA maximum allowed level of endotoxin is 5 EU per dose of drug per kg of subject body weight. See Guideline on Validation of the Limulus Amebocyte Lysate Test as an End-Product Endotoxin Test for Human and Animal Parenteral Drugs, Biological Products and Medical Devices, U.S. Dept. of Health & Human Services, FDA, December 1987. For example, for a standard 70 kg human subject, the maximum allowable endotoxin levels would be 350 EU (e.g., 5 EU multiplied by 70 kg). Based on the conversion, that would be equivalent to about 35 ng of endotoxin. Therefore, if the target antibody dose was 3 mg/kg and the subject weighed 70 kg, the correct antibody dosage would be 210 mg of antibody. Thus, for this circumstance, the maximum allowable endotoxin level for the antibody would be 350 EU/210 mg of antibody, or 1.67 EU/mg of anti-M-CSF antibody (i.e., or about 1.7 EU/mg of anti-M-CSF antibody). Accordingly, if dosing goes up, then the maximal allowable amount of endotoxin in the antibody composition will necessarily have to go down.

In preferred embodiments, the methods described herein can yield a composition comprising at least one M-CSF antibody that is substantially free of endotoxin.

As used herein, the term “substantially free of endotoxin” means that the concentration of endotoxins in an anti-M-CSF antibody composition is less than the amount permitted by the Food & Drug Administration (“FDA”) or an equivalent agency in protein compositions to be administered to humans or other animals as drugs. See Guideline on Validation of the Limulus Amebocyte Lysate Test as an End-Product Endotoxin Test for Human and Animal Parenteral Drugs, Biological Products, and Medical Devices, FDA, December (1987). Therefore, the endotoxin concentration is preferred to be 1) less than about 5 endotoxin units (EU) per dose per kilogram body weight when administered intravenously in a one-hour period and/or 2) less than about 1.7 EU/mg anti-M-CSF antibody.

In other embodiments, the methods described herein can yield a composition comprising at least one M-CSF antibody having a concentration of endotoxin of less than about 1.7 endotoxin unit per milligram of anti-M-CSF antibody (EU/mg) due to the particular antibody preparation and purification methods employed.

For example, the present invention provides a method of reducing the amount of endotoxin in a composition comprising at least one antibody comprising an amino acid sequence that is at least 90% identical to a light chain amino acid sequence shown in SEQ ID NO: 4, and further comprising an amino acid sequence that is at least 90% identical to a heavy chain amino acid sequence shown in SEQ ID NO: 2, wherein the antibody binds to human M-CSF, the method comprising contacting the composition with an affinity chromatography resin that binds to the antibody; eluting the antibody from the affinity chromatography resin to form an affinity chromatography eluent comprising the antibody; contacting the affinity chromatography eluent with an ion-exchange resin that binds to the antibody; and eluting the antibody from the ion-exchange resin, wherein the antibody is substantially free of endotoxin.

The aforementioned method, or any other methods or processes recited herein, can be performed in the order of the described steps or it may optionally be performed by varying the order of the steps or even repeating one or more of the steps. In one embodiment, the method of reducing the amount of endotoxin in a composition is performed in the order of the described steps. In some embodiments, the affinity chromatography resin contacting, washing and eluting steps are repeated in the same order more than one time before contacting the affinity chromatography eluent with the ion-exchange resin. The method can also include a filtering step using, for example, a 0.1 micron, 0.22 micron, or 0.44 micron filter, that can be performed on either one or more of the eluents removed after each resin binding.

In other embodiments, the present invention provides a method of reducing the amount of endotoxin in a composition comprising at least one antibody comprising an amino acid sequence that is at least 90% identical to a light chain amino acid sequence shown in SEQ ID NO: 4, and further comprising an amino acid sequence that is at least 90% identical to a heavy chain amino acid sequence shown in SEQ ID NO: 2, wherein the antibody binds to human M-CSF, the method comprising contacting the composition with an affinity chromatography resin that binds to the antibody; washing the resin; eluting the antibody from the affinity chromatography resin to form an affinity chromatography eluent comprising the antibody; contacting the affinity chromatography eluent with an ion-exchange resin that binds to the antibody; washing the resin; and eluting the antibody from the ion-exchange resin, wherein the antibody is substantially free of endotoxin.

In still other embodiments, the present invention provides a method of purifying a monoclonal IgG antibody comprising contacting the antibody with an affinity chromatography resin that binds to the antibody; washing the affinity chromatography resin with a wash solution comprising phosphate ions and chloride ions; washing the affinity chromatography resin with a wash solution comprising acetate ions at pH 5.5; eluting the antibody from the affinity chromatography resin to form an affinity chromatography eluent comprising the antibody; contacting the affinity chromatography eluent with an ion-exchange resin that binds to the antibody; and eluting the antibody from the ion-exchange resin.

### Affinity Chromatography

In certain instances, the steps of contacting the composition with affinity chromatography resin, washing and eluting the antibody from the affinity chromatography resin can be repeated more than one time before contacting the first eluent with an ion-exchange resin. In one embodiment, the affinity chromatography resin comprises a recombinant Protein A (“rProteinA”) resin. One example of a suitable recombinant Protein A resin is rProteinA Sepharose FF® resin (Amersham, Piscataway, N.J.). In another embodiment, a suitable affinity chromatography resin would comprise a protein G chromatography resin. In other embodiments, a suitable affinity chromatography resin comprises a mixed Protein A/Protein G resin. In other embodiments, a suitable affinity chromatography resin comprises a hydrophobic charge induction resin that comprises a 4-mercaptoethylpyridine ligand such as a MEP HyperCel® resin (BioSepra, Cergy, Saint Christopher, France).

### Ion-Exchange Chromatography

In some embodiments, it is preferred that the ion-exchange resin comprises an anion-exchange resin. As will be known by the person skilled in the art, ion exchangers may be based on various materials with respect to the matrix as well as to the attached charged groups. For example, the following matrices may be used, in which the materials mentioned may be more or less crosslinked: agarose based (such as Sepharose CL-6B®, Sepharose Fast Flow® and Sepharose High Performance®), cellulose based (such as DEAE Sephacel®), dextran based (such as Sephadex®), silica based and synthetic polymer based. For the anion exchange resin, the charged groups, which are covalently attached to the matrix, may, for example, be diethylaminoethyl, quaternary aminoethyl, and/or quaternary ammonium. It is preferred that the anion-exchange resin comprises a quaternary amine group. An exemplarily anion-exchange resin that has a quaternary amine group for binding the anti-M-CSF antibody is a Q Sepharose® resin (Amersham, Piscataway, N.J.).

In other aspects, if the endotoxin levels are higher than desired after subjecting the composition to the aforementioned ion-exchange chromatography step (e.g., anion exchange), the composition may be further subjected to a second ion-exchange step, for example, by contacting the compositions with a cation exchange resin and followed by a wash step, then elution from the ion-exchange resin. In preferred embodiments, the cation exchange resin comprises a sulfonic group for binding. An exemplary cation exchange resin is an SP Sepharose® resin FF (Amersham, Piscataway, N.J.).

### Finishing Step

The endotoxin amount may be further reduced by subjecting the sample to a step of concentrating and/or dialyzing the ion-exchange resin eluent. For example, after subjecting the ion-exchange eluent to another purification step via filtering through a 0.22 micron filter that can comprise polyether sulfone (PES), the ion-exchange eluent is preferably desalinated (i.e., dialysed) and optionally concentrated. The change in buffer and concentration of anti-M-CSF antibody can be performed by a combined process. It is contemplated that the diafiltration and concentration may be performed as two separate steps. However, in order to reduce unnecessary loss of the antibody, it is preferred to perform the dialysis and concentration by the method of diafiltration in one combined step.

Finally, the concentrated and dialyzed composition can be passed through one or more additional filter steps comprising an anion exchange functional group, such as a Millipore Intercept (Q Sepharose®) filter, for additional purification. In certain embodiments, the concentrated and dialyzed composition is passed through two anion charged filters (e.g., two Millipore Intercept (Q Sepharose®) filters) connected in tandem. Afterwards, the filtered liquid anti-M-CSF antibody composition is substantially free of endotoxin.

In certain embodiments, viral removal may also be carried out at any point during the method that is expedient. Viral removal may be accomplished by low pH inactivation (pH 3.5 to 3.7 for 30 to 90 minutes) or by filtration (e.g., nanofiltration) using membranes such as Pall DV 20™ membranes or Planova 15™ or 20N filters.

The foregoing description of the method of decreasing the amount of endotoxin in a composition described the steps as being sequential; however, those skilled in the art will understand that, in certain embodiments, some of the steps may be performed in a different order or simultaneously, so long as the amount of endotoxin is reduced. For example, the first affinity chromatography step may be substituted with the ion-exchange chromatography step so that the ion-exchange chromatography is performed first and the affinity chromatography step is performed in the next step.

Accordingly, in one embodiment, the present invention provides a composition comprising anti-M-CSF antibodies, wherein the composition is substantially free of endotoxin. In another embodiment, the composition has a concentration of endotoxin that is less than about 1.7 endotoxin units per milligram of M-CSF antibody (EU/mg).

In another embodiment, the present invention provides a composition comprising anti-M-CSF antibodies, wherein the composition has a concentration of endotoxin of less than about 1.6 EU/mg, and in other embodiments, less than about 1.5 EU/mg, and in other embodiments, less than about 1.4 EU/mg, and in other embodiments, less than about 1.3 EU/mg, and in other embodiments, less than about 1.2 EU/mg, and in other embodiments, less than about 1.1 EU/mg, and in other embodiments, less than about 1.0 EU/mg, and in other embodiments, less than about 0.09 EU/mg, and in other embodiments, less than about 0.08 EU/mg, and in other embodiments, less than about 0.05 EU/mg, and in other embodiments, less than about 0.04 EU/mg.

In another embodiment, the present invention provides a composition comprising anti-M-CSF antibodies, wherein the composition has a concentration of endotoxin ranging from about 0.001 EU/mg to about 1.6 EU/mg, and in other embodiments, ranging from about 0.005 to about 1.0 EU/mg, and in other embodiments, ranging from about 0.01 to about 0.5 EU/mg, and in other embodiments, ranging from about 0.02 to about 0.4 EU/mg, and in other embodiments, ranging from about 0.03 to about 0.3 EU/mg, and in other embodiments, ranging from about 0.04 to about 0.2 EU/mg, and in other embodiments, ranging from about 0.05 to about 0.1 EU/mg.

In some embodiments, the composition can be provided in a lyophilized format or it can optionally be provided in a liquid format. When the anti-M-CSF antibody composition is in a lyophilized format, the composition will be, in one embodiment, substantially free of endotoxin after reconstitution into a liquid composition. When referring to concentrations of endotoxin per milliliter of composition for lyophilized formats, it is intended that the reference concentration be used to describe the lyophilized composition after reconstitution.

Thus, in some aspects, the present invention provides compositions comprising M-CSF antibodies, wherein the composition has a concentration of endotoxin of less than about 3.0 endotoxin units per milliliter (EU/mL), and in another embodiment, less than about 1.0 EU/mL, and in another embodiment, less than about 0.5 EU/mL. In other embodiments, the present invention provides compositions comprising M-CSF antibodies, wherein the composition has a concentration of endotoxin that ranges from about 0.001 EU/mL to about 3.0 EU/mL, and in another embodiment, from about 0.01 EU/mL to about 3.0 EU/mL, and in another embodiment, from about 0.1 EU/mL to about 3.0 EU/mL, and in other embodiments, from about 0.5 EU/mL to about 3.0 EU/mL.

In one embodiment, when the anti-M-CSF antibody composition is intended for administration to a subject, the composition has an endotoxin concentration of less than about 0.5 endotoxin units (EU) per dose per kilogram body weight when administered intravenously over a one-hour period, and in other embodiments, the composition has an endotoxin concentration of less than about 0.1 endotoxin units (EU) per dose per kilogram body weight when administered intravenously over a one-hour period. In another embodiment, when the anti-M-CSF antibody composition is intended for administration to a subject, the composition has an endotoxin concentration that ranges from about 0.01 to about 0.5 EU per dose per kilogram body weight when administered intravenously over a one-hour period, and in still other embodiments, the endotoxin concentration ranges from about 0.05 to about 0.5 EU per dose per kilogram body weight when administered intravenously over a one-hour period.

Ranges intermediate to the above-recited endotoxin concentrations are also intended to be part of this invention. For example, ranges of values using a combination of any of the above-recited values as upper and/or lower limits are intended to be included.

### Endotoxin Assays

Limulus amebocyte lysate is an aqueous extract of the blood cells (amebocytes) of the North American horseshoe crab, Limulus polyphemus, which reacts with the lipopolysaccharide (LPS) moiety of bacterial endotoxin. See Novitsky, T., Annals of the New York Academy of Sciences 851:416-421 (1998). The active components of the reagent consist of a number of proteins, including enzymes (serine proteases) and ions. The enzymes, factors C and B and the proclotting enzyme, act in a cascade sequence. Factor C is activated by endotoxin's lipopolysaccharide, in turn activating factor B, which in turn activates the proclotting enzyme. The activated proclotting enzyme, referred to as the clotting enzyme of protein, in turn cleaves a protein termed coagulogen. Cleaved coagulogen reconfigures to an insoluble form termed coagulin. The coagulin self-aggregates to form a turbid gel.

One of skill in the art will understand how to determine the amounts of endotoxin in a given composition. For example, the FDA and USP have recognized the validity of various approaches to using one assay termed “Limulus Amebocyte Lysate” (LAL) for endotoxin testing. Among others, there are three methods available for endotoxin testing: (i) the gel-clot; (ii) the turbidimetric (spectrophotometric); and (iii) the chromogenic assay.

### The Gel-Clot LAL Assay

In one embodiment, the formation of a solid gel, or “gel clot”, is used as an end point for the LAL assay. If a purified LPS standard is used and incubation time and temperature are controlled, endotoxin concentration can be determined by observing the highest dilution exhibiting a solid gel clot. The Gel-Clot LAL assay can be performed by adding an equal volume of (e.g., 0.1 milliliter) sample dilution (20-, 10- or 2-fold series) to an equal volume of (e.g., 0.1 milliliter) of LAL reagent to in endotoxin-free 10×75 mm glass tubes and then incubating the tubes at 37° C. for 60 minutes.

The tubes are then turned over. If the clot remains at the bottom of the tube, it is considered positive for the presence of endotoxin. If liquid runs down the tube, it is considered negative for endotoxin at that dilution. Based on the dilution used and the behavior of positive controls, endotoxin levels can then be calculated within a particular range. See Novitsky, T., Annals of the New York Academy of Sciences 851:416-421 (1998).

Turbidimetric (spectrophotometric) LAL Assay

A second LAL assay form measures the turbidity (with a spectrophotometer, nephelometer, or optical reader) of the reaction and can be either an end point assay (fixed incubation time) or kinetic (rate of increase of turbidity) assay. See Novitsky, T., et al., J. Clin. Microbiol. 20: 211-216 (1985).

### Kinetic-Chromogenic LAL Assay

The Kinetic-Chromogenic Assay is a sensitive and inexpensive LAL assay. The Kinetic-Chromogenic Assay involves the use of a modified LAL reagent, which incorporates a chromogenic substrate. See Lindsay, G. et al., J. Clin. Microbiol. 27(5): 947-951 (1989). The substrate contains a small peptide that includes the cleavage site of coagulin and the chromophore paranitroaniline. This assay can also be either end point or kinetic and generally employs a spectrophotometer with a typical wavelength of 405 nm. Sensitivity of these assays based on a standard endotoxin reference varies from 0.03 endotoxin units per ml (gel clot method) to about 0.001 EU/ml with the kinetic turbidimetric/chromogenic methods. (One EU is equal to about 0.1 nanogram of purified Escherichia coli O113:H10:K(−)LPS.) One additional variation of the end point chromogenic assay involves conversion of released pNA to its diazo derivative with a reading at 545 nm. See Novitsky, T., Annals of the New York Academy of Sciences 851:416-421 (1998). An additional advantage is that the Kinetic-Chromogenic Assay provides quantitative data for use in trend analysis and process monitoring. Therefore, in preferred embodiments, the present invention utilizes a chromogenic LAL assay (e.g., a Cambrex Kinetic-Quantitative Chromogenic LAL assay) to determine the amounts of endotoxin in the compositions described herein. One embodiment of this particular assay is described in greater detail in Example 10.

In certain embodiments, the presence of endotoxin is determined using an endotoxin assay having a limit of detection of at least about 0.03 EU/mL. In other embodiments, the presence of endotoxin is determined using an endotoxin assay having a limit of detection of at least about 0.001 EU/mL.

In other embodiments, the presence of endotoxin is determined by a chromogenic LAL assay; wherein the antibody is 8.10.3F; wherein the endotoxin level is from about 0.04 to about 1 EU/mg. In other embodiments, the presence of endotoxin is determined by a chromogenic LAL assay; wherein the antibody is 8.10.3F; wherein the endotoxin level is from about 0.5 to about 3 EU/ml.

### Methods of Producing Anti-M-CSF Antibodies and Antibody Producing Cell Lines:

Antibodies in accordance with the invention can be prepared through the utilization of a transgenic mouse that has a substantial portion of the human antibody producing genome inserted, but that is rendered deficient in the production of endogenous, murine, antibodies. Such mice, then, are capable of producing human immunoglobulin molecules and antibodies and are deficient in the production of murine immunoglobulin molecules and antibodies. Technologies utilized for achieving the same are discussed below.

It is possible to produce transgenic animals (e.g., mice) that are capable, upon immunization, of producing a full repertoire of human antibodies in the absence of endogenous immunoglobulin production. In particular, however, one embodiment of transgenic production of mice and antibodies therefrom is disclosed in U.S. Published Application No. 20050059113 to Bedian, et al. Through use of such technology, antibodies that bind to M-CSF and hybridomas producing such antibodies can be prepared.

Human antibodies avoid potential problems associated with antibodies that possess murine or rat variable and/or constant regions. The presence of such murine or rat derived proteins can lead to the rapid clearance of the antibodies or can lead to the generation of an immune response against the antibody by a subject that receives administration of such antibodies.

For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen (e.g., CTLA-4) challenge. See, e.g., Jakobovits et al, Proc. Natl. Acad. Sci. USA, 90:2551 (1993); Jakobovits et al, Nature, 362:255-258 (1993); Bruggermann et al., Year in Immuno., 7:33 (1993); and Duchosal et al., Nature 355:258 (1992). Human antibodies can also be derived from phage-display libraries (Hoogenboom et al., J. Mol. Biol., 227:381 (1991); Marks et al., J. Mol. Biol., 222:581-597 (1991); Vaugan et al., Nature Biotech 14:309 (1996)).

In some embodiments, human antibodies are produced by immunizing a non-human animal comprising in its genome some or all of human immunoglobulin heavy chain and light chain loci with an M-CSF antigen. In a preferred embodiment, the non-human animal is a, XENOMOUSE™ animal (Abgenix Inc., Fremont, Calif.). Another non-human animal that may be used is a transgenic mouse produced by Medarex (Medarex, Inc., Princeton, N.J.).

In some embodiments, human anti-M-CSF antibodies can be produced by immunizing a non-human transgenic animal, e.g., XENOMOUSE™ mice, whose genome comprises human immunoglobulin genes so that the recombinant mouse produces human antibodies. XENOMOUSE™ mice are engineered mouse strains that comprise large fragments of human immunoglobulin heavy chain and light chain loci and are deficient in mouse antibody production. XENOMOUSE™ mice produce an adult-like human repertoire of fully human antibodies and generate antigen-specific human antibodies. In some embodiments, the XENOMOUSE™ mice contain approximately 80% of the human antibody V gene repertoire through introduction of megabase sized, germline configuration yeast artificial chromosome (YAC) fragments of the human heavy chain loci and kappa light chain loci. In other embodiments, XENOMOUSE™ mice further contain approximately all of the lambda light chain locus. See, e.g., Green et al., Nature Genetics 7:13-21 (1994) and U.S. Pat. Nos. 5,916,771, 5,939,598, 5,985,615, 5,998,209, 6,075,181, 6,091,001, 6,114,598, 6,130,364, 6,162,963 and 6,150,584. See also WO 91/10741, WO 94/02602, WO 96/34096, WO 96/33735, WO 98/16654, WO 98/24893, WO 98/50433, WO 99/45031, WO 99/53049, WO 00/09560, and WO 00/037504.

In some embodiments, the non-human animal comprising human immunoglobulin genes are animals that have a human immunoglobulin “minilocus”. In the minilocus approach, an exogenous Ig locus is mimicked through the inclusion of individual genes from the Ig locus. Thus, one or more V_{H} genes, one or more D_{H} genes, one or more J_{H} genes, a mu constant domain, and a second constant domain (preferably a gamma constant domain) are formed into a construct for insertion into an animal. This approach is described, inter alia, in U.S. Pat. Nos. 5,545,807, 5,545,806, 5,569,825, 5,625,126, 5,633,425, 5,661,016, 5,770,429, 5,789,650, 5,814,318, 5,591,669, 5,612,205, 5,721,367, 5,789,215, and 5,643,763.

Therefore, in some embodiments, human antibodies can be produced by immunizing a non-human animal comprising in its genome some or all of human immunoglobulin heavy chain and light chain loci with an M-CSF antigen.

In some embodiments, the M-CSF antigen is isolated and/or purified M-CSF. In a preferred embodiment, the M-CSF antigen is human M-CSF. In some embodiments, the M-CSF antigen is a fragment of M-CSF. In some embodiments, the M-CSF fragment comprises at least one epitope of M-CSF. In other embodiments, the M-CSF antigen is a cell that expresses or overexpresses M-CSF or an immunogenic fragment thereof on its surface. In still other embodiments, the M-CSF antigen is an M-CSF fusion protein. M-CSF can be purified from natural sources using known techniques. In addition, recombinant M-CSF protein is commercially available.

In a preferred embodiment, the non-human animal is a XENOMOUSE™ animal (Abgenix Inc., Fremont, Calif.). Another non-human animal that may be used is a transgenic mouse produced by Medarex (Medarex, Inc., Princeton, N.J.).

Immunization of animals can be by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow and Lane, supra, and U.S. Pat. No. 5,994,619. In a preferred embodiment, the M-CSF antigen is administered with an adjuvant to stimulate the immune response. Exemplary adjuvants include complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule can involve two or more administrations of the polypeptide, spread out over several weeks.

After immunization of an animal with an M-CSF antigen, antibodies and/or antibody-producing cells can be obtained from the animal. In some embodiments, anti-M-CSF antibody-containing serum is obtained from the animal by bleeding or sacrificing the animal. The serum may be used as it is obtained from the animal, an immunoglobulin fraction may be obtained from the serum, or the anti-M-CSF antibodies may be purified from the serum.

In some embodiments, antibody-producing immortalized cell lines are prepared from cells isolated from the immunized animal. After immunization, the animal is sacrificed and lymph node and/or splenic B cells are immortalized. Methods of immortalizing cells include, but are not limited to, transfecting them with oncogenes, infecting them with an oncogenic virus, cultivating them under conditions that select for immortalized cells, subjecting them to carcinogenic or mutating compounds, fusing them with an immortalized cell, e.g., a myeloma cell, and inactivating a tumor suppressor gene. See, e.g., Harlow and Lane, supra. In a preferred embodiment, the immunized animal is a non-human animal that expresses human immunoglobulin genes and the splenic B cells are fused to a myeloma cell line from the same species as the non-human animal. In a more preferred embodiment, the immunized animal is a XENOMOUSE™ animal and the myeloma cell line is a non-secretory mouse myeloma. In an even more preferred embodiment, the myeloma cell line is P3-X63-AG8-653. If fusion with myeloma cells is used, the myeloma cells preferably do not secrete immunoglobulin polypeptides (a non-secretory cell line). Immortalized cells are screened using M-CSF, a portion thereof, or a cell expressing M-CSF. In a preferred embodiment, the initial screening is performed using an enzyme-linked immunoassay (ELISA) or a radioimmunoassay. An example of ELISA screening is provided in WO 00/37504.

Anti-M-CSF antibody-producing cells, e.g., hybridomas, are selected, cloned and further screened for desirable characteristics, including robust growth, high antibody production and desirable antibody characteristics, as discussed further below. Hybridomas can be expanded in vivo in syngeneic animals, in animals that lack an immune system, e.g., nude mice, or in cell culture in vitro. Methods of selecting, cloning and expanding hybridomas are well known to those of ordinary skill in the art.

As will be appreciated, antibodies in accordance with the present invention can be recombinantly expressed in cell lines other than hybridoma cell lines. Nucleic acid sequences encoding the cDNAs or genomic clones for the particular antibodies can be used for transformation of a suitable mammalian or nonmammalian host cells.

The present invention also encompasses nucleic acid molecules encoding anti-M-CSF antibodies. In some embodiments, different nucleic acid molecules encode a heavy chain and a light chain of an anti-M-CSF immunoglobulin. In other embodiments, the same nucleic acid molecule encodes a heavy chain and a light chain of an anti-M-CSF immunoglobulin. In one embodiment, the nucleic acid encodes an anti-M-CSF antibody of the invention.

A nucleic acid molecule encoding the heavy or entire light chain of an anti-M-CSF antibody or portions thereof can be isolated from any source that produces such antibody. In various embodiments, the nucleic acid molecules are isolated from a B cell isolated from an animal immunized with anti-M-CSF or from an immortalized cell derived from such a B cell that expresses an anti-M-CSF antibody. Methods of isolating mRNA encoding an antibody are well-known in the art. See, e.g., Sambrook, et al., Molecular Cloning 3rd Ed. Vol. 3 (1989). The mRNA may be used to produce cDNA for use in the polymerase chain reaction (PCR) or cDNA cloning of antibody genes. In a preferred embodiment, the nucleic acid molecule is isolated from a hybridoma that has as one of its fusion partners a human immunoglobulin-producing cell from a non-human transgenic animal. In an even more preferred embodiment, the human immunoglobulin producing cell is isolated from a XENOMOUSE™ animal. In another embodiment, the human immunoglobulin-producing cell is from a non-human, non-mouse transgenic animal, as described above. In another embodiment, the nucleic acid is isolated from a non-human, non-transgenic animal. The nucleic acid molecules isolated from a non-human animal may be used, e.g., for humanized antibodies.

In some embodiments, a nucleic acid encoding a heavy chain of an anti-M-CSF antibody of the invention can comprise a nucleotide sequence encoding a V_{H} domain of the invention joined in-frame to a nucleotide sequence encoding a heavy chain constant domain from any source. Similarly, a nucleic acid molecule encoding a light chain of an anti-M-CSF antibody of the invention can comprise a nucleotide sequence encoding a V_{L} domain of the invention joined in-frame to a nucleotide sequence encoding a light chain constant domain from any source.

In a further aspect of the invention, nucleic acid molecules encoding the variable domain of the heavy (V_{H}) and light (V_{L}) chains are “converted” to full-length antibody genes. In one embodiment, nucleic acid molecules encoding the V_{H} or V_{L} domains are converted to full-length antibody genes by insertion into an expression vector already encoding heavy chain constant (C_{H}) or light chain (C_{L}) constant domains, respectively, such that the V_{H} segment is operatively linked to the C_{H} segment(s) within the vector, and the V_{L} segment is operatively linked to the C_{L} segment within the vector. In another embodiment, nucleic acid molecules encoding the V_{H} and/or V_{L} domains are converted into full-length antibody genes by linking, e.g., ligating, a nucleic acid molecule encoding a V_{H} and/or V_{L} domains to a nucleic acid molecule encoding a C_{H} and/or C_{L} domain using standard molecular biological techniques. Nucleic acid sequences of human heavy and light chain immunoglobulin constant domain genes are known in the art. See, e.g., Kabat et al., Sequences of Proteins of Immunological Interest, 5th Ed., NIH Publ. No. 91-3242,1991. Nucleic acid molecules encoding the full-length heavy and/or light chains may then be expressed from a cell into which they have been introduced and the anti-CTLA-4 antibody isolated.

The present invention also provides vectors comprising nucleic acid molecules that encode the heavy chain of an anti-M-CSF antibody of the invention or an antigen-binding portion thereof. The invention also provides vectors comprising nucleic acid molecules that encode the light chain of such antibodies or antigen-binding portion thereof. The invention further provides vectors comprising nucleic acid molecules encoding fusion proteins, modified antibodies, antibody fragments, and probes thereof.

In some embodiments, the anti-M-CSF antibodies, or antigen-binding portions of the invention are expressed by inserting DNAs encoding partial or full-length light and heavy chains, obtained as described above, into expression vectors such that the genes are operatively linked to necessary expression control sequences such as transcriptional and translational control sequences. Expression vectors include plasmids, retroviruses, adenoviruses, adeno-associated viruses (AAV), plant viruses such as cauliflower mosaic virus, tobacco mosaic virus, cosmids, YACs, EBV derived episomes, and the like. The antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors. In a preferred embodiment, both genes are inserted into the same expression vector. The antibody genes are inserted into the expression vector by standard methods (e.g., ligation of complementary restriction sites on the antibody gene fragment and vector, or blunt end ligation if no restriction sites are present).

A convenient vector is one that encodes a functionally complete human C_{H} or C_{L} immunoglobulin sequence, with appropriate restriction sites engineered so that any V_{H} or V_{L} sequence can easily be inserted and expressed, as described above. In such vectors, splicing usually occurs between the splice donor site in the inserted J region and the splice acceptor site preceding the human C domain, and also at the splice regions that occur within the human C_{H} exons. Polyadenylation and transcription termination occur at native chromosomal sites downstream of the coding regions. The recombinant expression vector also can encode a signal peptide that facilitates secretion of the antibody chain from a host cell. The antibody chain gene may be cloned into the vector such that the signal peptide is linked in-frame to the amino terminus of the immunoglobulin chain. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviruses (such as retroviral LTRs), cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. No. 5,168,062, U.S. Pat. No. 4,510,245 and U.S. Pat. No. 4,968,615. Methods for expressing antibodies in plants, including a description of promoters and vectors, as well as transformation of plants is known in the art. See, e.g., U.S. Pat. No. 6,517,529. Methods of expressing polypeptides in bacterial cells or fungal cells, e.g., yeast cells, are also well known in the art.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in DHFR-host cells with methotrexate selection/amplification), the neomycin resistance gene (for G418 selection), and the glutamine synthetase gene.

Nucleic acid molecules encoding anti-M-CSF antibodies and vectors comprising these nucleic acid molecules can be used for transformation of a suitable mammalian, plant, bacterial or yeast host cell. Antibodies of the invention can be produced transgenically through the generation of a mammal or plant that is transgenic for the immunoglobulin heavy and light chain sequences of interest and production of the antibody in a recoverable form therefrom.

Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455. The transformation procedure used depends upon the host to be transformed. Methods for introduction of heterologous polynucleotides into mammalian cells are well known in the art and include, but are not limited to, dextran-mediated transfection, calcium phosphate precipitation, polybrene mediated transfection, protoplast fusion, electroporation, particle bombardment, encapsulation of the polynucleotide(s) in liposomes, peptide conjugates, dendrimers, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, NS0 cells, SP2 cells, HeLa cells, baby hamster kidney (BHK) cells, monkey kidney cells (COS), human hepatocellular carcinoma cells (e.g., Hep G2), and a number of other cell lines. Non-mammalian cells, including but not limited to, bacterial (e.g., E. coli and Streptomyces species), yeast (e.g., Schizosaccharomyces pombe, Saccharomyces cerevisiae and Pichia pastoris), insect (e.g., Sf9 cells), and plants can also be used to express recombinant antibodies.

Production of recombinant antibodies in Chinese hamster ovary (CHO) cells is the most widely used mammalian expression system, particularly for production of antibodies. The most commonly used CHO expression system is based on the use of CHO cells deficient in the production of endogenous dihydrofolate reductase (DHFR) coupled with a DHFR gene amplification system. These DHFR⁻ CHO cells are transfected with either a single plasmid containing both antibody genes and afunctional DHFR gene or two plasmids with the DHFR gene contained on a separate plasmid from the antibody (heavy or light chain gene) cassettes. In other embodiments, the DHFR gene is on the plasmid that encodes either the heavy or light chain.

Transfected cells are selected in increasing concentrations of the drug methotrexate. Survival on high concentrations of methotrexate (1 to 10 μM) is associated with gene amplification of the DHFR gene during integration into the host chromosome or integration into active regions of the chromosome. During the DHFR gene amplification step, the antibody genes are also coamplified and integrated into the host chromosome.

The expression methods are selected by determining which system generates the highest expression levels and produce antibodies with constitutive M-CSF binding properties. Further, expression of antibodies of the invention (or other moieties therefrom) from production cell lines can be enhanced using a number of known techniques. For example, the glutamine synthetase and DHFR gene expression systems are common approaches for enhancing expression under certain conditions. High expressing cell clones can be identified using conventional techniques, such as limited dilution cloning and Microdrop technology. The Glutamine Synthetase system is discussed in whole or part in connection with European Patent Nos. 0 216 846, 0 256 055, and 0 323 997 and European Patent Application No. 89303964.4.

In connection with the transgenic production in mammals, antibodies can also be produced in, and recovered from, the milk of goats, cows, or other mammals. See, e.g., U.S. Pat. Nos. 5,827,690, 5,756,687, 5,750,172, and 5,741,957.

When recombinant expression vectors encoding anti-M-CSF antibody genes are introduced into host cells, the antibodies are produced by culturing the host cells for a period of time sufficient to allow for expression of the antibody in the host cells or, more preferably, secretion of the antibody into the culture medium in which the host cells are grown. The antibodies may be present in the culture medium, whole cells, in a cell lysate, or in a partially purified or substantially pure form. The antibodies expressed in cell lines as described above may be purified and/or isolated from the associated cellular material. Purification is performed in order to eliminate other cellular components or other contaminants, e.g. other cellular nucleic acids or proteins, by standard techniques, including alkaline/SDS treatment, column chromatography and others well known in the art. See Ausubel, F., et al., ed. Current Protocols in Molecular Biology, Greene Publishing and Wiley Interscience, New York (1987). In one embodiment, the antibodies can be recovered from the culture medium using protein purification methods, including the purification methods described in the Examples herein.

In the present invention, it is possible that anti-M-CSF antibodies expressed by different cell lines or in transgenic animals will have different glycosylation patterns from each other. However, all of the anti-M-CSF antibodies encoded by the nucleic acids and amino acids provided herein are considered part of the instant invention, regardless of their glycosylation pattern or modification or deletion thereof. Thus, for purposes of the present invention, the anti-M-CSF antibodies may be glycosylated or non-glycosylated. When the anti-M-CSF antibodies are glycosylated they may have any possible glycosylation pattern. Site directed mutagenesis of the antibody CH2 domain to eliminate glycosylation is also encompassed by the present invention in order to prevent changes in either the immunogenicity, pharmacokinetic, and/or effector functions resulting from non-human glycosylation.

As used herein, the term “glycosylation” means the pattern of carbohydrate units that are covalently attached to an antibody. When it is said that the anti-M-CSF antibodies herein have a particular glycosylation pattern, it is meant that the majority of the referenced anti-M-CSF antibodies have that particular glycosylation pattern. In other aspects, when it is said that the anti-M-CSF antibodies herein have a particular glycosylation pattern, it is meant that greater than or equal to 75%, 90%, 95%, or 99% of the referenced anti-M-CSF antibodies have that particular glycosylation pattern.

The anti-M-CSF antibodies of the present invention also encompass glycosylation variants thereof (e.g., by insertion of a glycosylation site or deletion of any glycosylation site by deletion, insertion or substitution of suitable amino acid residues).

Glycosylation of polypeptides is typically either N-linked or O-linked. Glycosylation of antibody polypeptides is typically N-linked and forms a biantennary structure. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in an antibody creates a potential glycosylation site.

The three distinct structures of biantennary glycans are designated “G0”, “G1” and “G2” having zero, one, or two, respectively, terminal galactose residues on the nonreducing end of the glycan. See Jefferis et al., Biochem. J., 268, 529-537 (1990). In some cases, the glycan structure may also have a fucose residue linked to an N-acetylglucosamine, which is covalently bonded to the asparagine amino acid (e.g., position 297) found in the antibody. When the fucose (F) is present, the biantennary glycan nomenclature is changed to “G0F”, “G1F”, or “G2F” depending upon: the number of terminal galactose residues. See Teillaud, Expert Opin. Biol. Ther., 5(Suppl.1):S1327 (2005). Furthermore, when the antibody contains both of the two heavy chains, the glycan nomenclature is repeated for each of the two heavy chains. Moreover, each heavy chain within one antibody may have the same glycosylation pattern or the two heavy chains may have differing glycosylation patterns. In certain embodiments, the anti-M-CSF antibodies have a glycosylation pattern that is selected from the group consisting of “G0F,G0F”; “G0F,G1F”; “G1F,G1F”; “G1F,G2F”; and mixtures thereof.

For example, in one embodiment, the anti-M-CSF antibody 8.10.3F described herein has a glycosylation pattern of “G0F,G0F” as reported in Example 10. The “G0F,G0F” glycoform is a species in which both heavy chains have the G0 glycan attached and each G0 glycan has a fucose (F) residue linked to an N-acetylglucosamine, which is covalently bonded to an asparagine amino acid at residue 297 found in the heavy chains of antibody 8.10.3F.

### Preparation of the Monoclonal Anti-M-CSF Antibody Formulations:

The anti-M-CSF antibody typically is formulated as a composition for parenteral administration to a subject. In one embodiment, the composition is a liquid pharmaceutical composition.

The compositions of the present invention involve one or more anti-M-CSF monoclonal antibodies of the invention in combination with pharmaceutically acceptable excipients, which comprise histidine and/or a chelating agent.

The term “pharmaceutical composition” refers to preparations which are in such form as to permit the biological activity of the active ingredients to be effective. “Pharmaceutically acceptable excipients” (vehicles, additives) are those, which can reasonably (i.e., safely) be administered to a subject to provide an effective dose of the active ingredient employed. The term “excipient” or “carrier” as used herein refers to an inert substance, which is commonly used as a diluent, vehicle, preservative, binder or stabilizing agent for drugs. As used herein, the term “diluent” refers to a pharmaceutically acceptable (safe and non-toxic for administration to a human) solvent and is useful for the preparation of the liquid formulations herein. Exemplary diluents include, but are not limited to, sterile water and bacteriostatic water for injection (BWFI).

In one embodiment, the liquid pharmaceutical composition comprises at least one antibody comprising an amino acid sequence that is at least 90%, 95% or 99% identical to a light chain sequence shown in SEQ ID NO: 4, and further comprising an amino acid sequence that is at least 90%, 95%, or 99% identical to a heavy chain amino acid sequence shown in SEQ ID NO: 2; and a chelating agent, wherein the antibody binds to human M-CSF and the composition is substantially free of endotoxin.

In another embodiment, the liquid pharmaceutical composition comprises at least one antibody comprising an amino acid sequence that is at least 90%, 95% or 99% identical to a light chain sequence shown in SEQ ID NO: 4, and further comprising an amino acid sequence that is at least 90%, 95%, or 99% identical to a heavy chain amino acid sequence shown in SEQ ID NO: 2; and a chelating agent, wherein the antibody binds to human M-CSF and the composition is substantially free of endotoxin and further comprising at least one or more pharmaceutically acceptable excipient that is chosen from buffers, tonicity agents, antioxidants, and surfactants.

In another embodiment, the liquid pharmaceutical composition comprises at least one antibody comprising a heavy chain amino acid sequence that comprises the variable region of SEQ ID NO: 2 and a light chain amino acid sequence that comprises the variable region SEQ ID NO: 4; and a chelating agent, wherein the antibody binds to human M-CSF and the composition is substantially free of endotoxin.

In another embodiment, the liquid pharmaceutical composition comprises at least one antibody comprising a human monoclonal IgG2 antibody having the heavy and light chain amino acid sequences of antibody 8.10.3F; and a chelating agent, wherein the antibody binds to human M-CSF and the composition is substantially free of endotoxin.

The concentration of the anti-M-CSF antibody in the liquid pharmaceutical compositions of the present invention is generally at least about 0.1 milligram per milliliter (mg/ml) or higher, at least about 1.0 mg/ml or higher, at least about 10 mg/ml or higher, at least about 20 mg/ml or higher, at least about 50 mg/ml or higher, at least about 100 mg/ml or higher, or at least about 200 mg/ml or higher. In certain embodiments, the concentration of the anti-M-CSF antibody generally ranges from about 0.1 mg/ml to about 200 mg/ml, from about 0.5 mg/ml to about 100 mg/ml, from about 1 mg/ml to about 50 mg/ml, from about 2.0 mg/ml to about 35 mg/ml, from about 5.0 mg/ml to about 25 mg/ml, or from about 7 mg/ml to about 15 mg/ml. In one embodiment, the concentration of the anti-M-CSF antibody in the liquid pharmaceutical compositions of the present invention is generally about 5 mg/ml, about 10 mg/ml, about 20 mg/ml, about 50 mg/ml, about 65 mg/ml, about 70 mg/ml, about 75 mg/ml, about 80 mg/ml, about 85 mg/ml, or about 100 mg/ml. In another embodiment, the concentration of the anti-M-CSF antibody in the liquid pharmaceutical composition ranges from about 1 mg/ml to about 50 mg/ml. In one embodiment, the concentration of the anti-M-CSF antibody in the liquid pharmaceutical composition is about 10 mg/ml. In another embodiment, the concentration of the anti-M-CSF antibody in the liquid pharmaceutical composition is about 75 mg/ml.

In another embodiment, the concentration of the anti-M-CSF antibody in the liquid pharmaceutical composition ranges from about 50 mg/ml to about 100 mg/ml. In some embodiments, higher antibody concentrations can be used where the composition is intended for subcutaneous delivery.

As used herein, the terms “chelating agent” generally refers to an excipient that can form at least one bond (e.g., covalent, ionic, or otherwise) to a metal ion. A chelating agent is typically a multidentate ligand that can be used in selected liquid compositions as a stabilizer to complex with species, which might promote instability. Often, compounds that can act as a chelating agent will have electron-rich functional groups. Suitable electron-rich functional groups include carboxylic acid groups, hydroxy groups and amino groups. Arrangement of these groups in aminopolycarboxylic acids, hydroxypolycarboxylic acids, hydroxyaminocarboxylic acids, and the like, result in moieties that have the capacity to bind metal.

However, the present invention is not intended to be limited to chelating agents primarily by the chelating agent's ability to form bonds with a metal ion. Therefore, the present invention is not intended to be limited by any specific mechanism by which the chelating agent acts in the formulations of the present invention and the excipients termed chelating agents herein may achieve their properties through mechanisms that are altogether unrelated to the chelating agent's ability to form bonds with a metal ion.

Chelating agents that are suitable for use in the present invention, include, but are not limited to, aminopolycarboxylic acids, hydroxyaminocarboxylic acids, N-substituted glycines, 2-(2-amino-2-oxoethyl) aminoethane sulfonic acid (BES), deferoxamine (DEF), citric acid, niacinamide, and desoxycholates. Examples of suitable aminopolycarboxylic acids include ethylenediaminetetraacetic acid (EDTA), diethylenetriamine pentaacetic acid 5 (DTPA), nitrilotriacetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), glutamic acid, and aspartic acid. Examples of suitable hydroxyaminocarboxylic acids include N-hydroxyethyliminodiacetic acid (HIMDA), N,N-bis-hydroxyethylglycine (bicine) and N-(trishydroxymethylmethyl) glycine (tricine). An example of a suitable N-substituted glycine is glycylglycine. An example of a suitable desoxycholate is sodium desoxycholate. Mixtures of two or more chelating agents are also encompassed by the present invention.

Chelating agents used in the invention can be present, where possible, as the free acid or free base form of the compound (e.g., referred to interchangeably herein as “EDTA” or “edetate”) or as a corresponding salt form (e.g., the corresponding acid addition salt or base addition salt, such as disodium edetate). Suitable acid addition salts, e.g., include alkali metal salts (e.g., sodium or potassium salts), alkaline earth metal salts (e.g., calcium salts), and salts can be prepared using other weakly bound metal ions. As is known in the art, the nature of the salt and the number of charges to be neutralized will depend on the number of carboxyl groups present and the pH at which the stabilizing chelating agent is supplied. As is also known in the art, chelating agents have varying strengths with which particular target ions are bound. By way of further illustration, suitable salts of EDTA include dipotassium edetate, disodium edetate, edetate calcium disodium, sodium edetate, trisodium edetate, and potassium edetate; and a suitable salt of deferoxamine (DEF) is deferoxamine mesylate (DFM).

Chelating agents used in the invention can be present as an anhydrous, solvated or hydrated form of the compound or corresponding salt. Where the chelating agent is in a solvated or hydrated form, it can be present in varying states of solvation or hydration (including, e.g., anhydrous, hydrated, dihydrated, and trihydrated forms). By way of further illustration, a suitable hydrate of EDTA is disodium EDTA dihydrate; and suitable forms of citric acid include anhydrous citric acid, citric acid monohydrate, and trisodium citrate-dihydrate.

Suitable chelating agents used in the antibody compositions of the present invention also include, for example, those that bind to metal ions in solution to render them unable to react with available O₂, thereby minimizing or preventing generation of hydroxyl radicals which are free to react with and degrade the antibody. Chelating agents can lower the formation of reduced oxygen species, reduce acidic species (e.g., deamidation) formation, reduce antibody aggregation, and/or reduce antibody fragmentation in the compositions of the present invention. Such chelating agents can reduce or prevent degradation of an antibody that is formulated without the protection of a chelating agent.

When a concentration of a chelating agent is referred to, it is intended that the recited concentration represent the molar concentration of the free acid or free base form of the chelating agent. For example, the concentration of chelating agent in certain liquid pharmaceutical compositions generally ranges from about 0.01 micromolar to about 50 millimolar, from about 1 micromolar to about 10.0 millimolar, from about 15 micromolar to about 5.0 millimolar, from about 0.01 millimolar to about 1.0 millimolar, or from about 0.03 millimolar to about 0.5 millimolar. In certain embodiments, the concentration of chelating agent in the liquid pharmaceutical composition can be about 0.01 millimolar, 0.02 millimolar, 0.027 millimolar, 0.03 millimolar, about 0.04 millimolar, about 0.05 millimolar, about 0.06 millimolar, about 0.07 millimolar, about 0.10 millimolar, about 0.20 millimolar, about 0.26 millimolar, about 0.27 millimolar, about 0.30 millimolar, about 0.31 millimolar, about 0.34 millimolar, about 0.40 millimolar, about 0.50 millimolar, or about 1.0 millimolar. In certain embodiments, the concentration of chelating agent is about 0.027 millimolar, about 0.05 millimolar, about 0.13 millimolar, or about 0.27 millimolar. In one embodiment, the concentration of chelating agent is about 0.05 millimolar. In another embodiment, the concentration of chelating agent is about 0.13 millimolar.

Unless stated otherwise, the concentrations listed herein are those concentrations at ambient conditions, (i.e., at 25° C. and atmospheric pressure). Ranges intermediate to the above-recited chelating agent concentrations are also intended to be part of this invention. For example, ranges of values using a combination of any of the above-recited values as upper and/or lower limits are intended to be included.

In one embodiment, the chelating agent is selected from the group consisting of EDTA, DTPA, DFM, and mixtures thereof. In another embodiment, the chelating is agent is DFM. In another embodiment, the chelating agent is EDTA. In another embodiment, the chelating agent is DTPA. In another embodiment, the liquid pharmaceutical composition comprises EDTA in an amount that generally ranges from about 0.01 micromolar to about 50 millimolar, from about 1 micromolar to about 20.0 millimolar, from about 15 micromolar to about 10.0 millimolar, from about 0.01 millimolar to about 5.0 millimolar, or from about 0.03 millimolar to about 1 millimolar. In certain embodiments, the concentration of EDTA in the liquid pharmaceutical composition can be about 0.01 millimolar, 0.02 millimolar, 0.027 millimolar, 0.03 millimolar, about 0.04 millimolar, about 0.05 millimolar, about 0.06 millimolar, about 0.07 millimolar, about 0.10 millimolar, about 0.20 millimolar, about 0.26 millimolar, about 0.27 millimolar, about 0.30 millimolar, about 0.31 millimolar, about 0.34 millimolar, about 0.40 millimolar, about 0.50 millimolar, or about 1.0 millimolar. In certain embodiments, the concentration of EDTA is about 0.027 millimolar, about 0.05 millimolar, about 0.13 millimolar, or about 0.27 millimolar. In one embodiment, the concentration of EDTA is about 0.05 millimolar. In another embodiment, the concentration of EDTA is about 0.13 millimolar.

As noted above, the compositions of the present invention optionally may further comprise a buffer in addition to a chelating agent. As used herein, the term “buffer” refers to an added composition that allows a liquid antibody formulation to resist changes in pH.

In certain embodiments, the added buffer allows a liquid antibody formulation to resist changes in pH by the action of its acid-base conjugate components. For example, a buffered formulation may be prepared by adding L-histidine-HCl (L-histidine-hydrochloride) and L-histidine in the appropriate amounts to arrive at a desired pH. However, in other embodiments, the added buffer allows a liquid antibody formulation to resist changes in pH by the action of its acid-base conjugate components. By way of a second example, a buffered formulation may be prepared by adding an acid, such as hydrochloric acid, and L-histidine in the appropriate amounts to arrive at a desired pH.

Examples of suitable buffers include, but are not limited to, acetate (e.g., sodium acetate), succinate (e.g., sodium succinate), gluconate, citrate (e.g., and other organic acid buffers, including, but not limited to, buffers such as amino acids (e.g., histidine), acetic acid, phosphoric acid and phosphates, ascorbate, tartartic acid, maleic acid, glycine, lactate, lactic acid, ascorbic acid, imidazoles, carbonic acid and bicarbonates, succinic acid, sodium benzoic acid and benzoates, gluconate, edetate (EDTA), acetate, malate, imidazole, tris, phosphate, and mixtures thereof. In one embodiment, the buffer is acetate.

In another embodiment, the buffer is histidine. The histidine starting material used to prepare the compositions of the present invention can exist in different forms. For example, the histidine can be an enantiomeric (e.g., L- or D-enantiomer) or racemic form of histidine, a free acid or free base form of histidine, a salt form (e.g., a monohydrochloride, dihydrochloride, hydrobromide, sulfate, or acetate salt) of histidine, a solvated form of histidine, a hydrated form (e.g., monohydrate) of histidine, or an anhydrous form of histidine. The purity of histidine base and/or salt used to prepare the compositions generally can be at least about 98%, at least about 99%, or at least about 99.5%. As used herein, the term “purity” in the context of histidine refers to chemical purity of histidine as understood in the art, e.g., as described in The Merck Index, 13th ed., O'Neil et al. ed. (Merck & Co., 2001).

When a concentration of a buffer is referred to, it is intended that the recited concentration represent the molar concentration of the free acid or free base form of the buffer. For example, the concentration of the buffer when present in certain liquid pharmaceutical compositions can range from about 0.1 millimolar (mM) to about 100 mM. In one embodiment, the concentration of the buffer is from about 1 mM to about 50 mM. In another embodiment, the concentration of the buffer is from about 5 mM to about 30 mM. In various embodiments, the concentration of the buffer is about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM or about 100 mM. In one embodiment, the concentration of histidine in the pharmaceutical composition is about 10 mM. In another embodiment, the pharmaceutical composition contains about 10 mM of L-histidine (in base form). In another embodiment, the concentration of histidine in the pharmaceutical composition is about 20 mM. In another embodiment, the pharmaceutical composition contains about 20 mM of L-histidine (in base form). Ranges intermediate to the above-recited histidine concentrations are also intended to be part of this invention. For example, ranges of values using a combination of any of the above-recited values as upper and/or lower limits are intended to be included.

In general, the buffer is used to maintain an acceptable pH level (which can affect antibody stability) in the liquid pharmaceutical composition. The liquid pharmaceutical composition typically is buffered to maintain a pH in the range of from about 4 to about 8; from about 4.5 to about 7; from about 5.0 to 6.5, or from about 5.3 to about 6.3. Ranges intermediate to the above-recited pH's are also intended to be part of this invention. For example, ranges of values using a combination of any of the above-recited values as upper and/or lower limits are intended to be included. In one embodiment, the liquid pharmaceutical composition is buffered to maintain a pH of about 5.5. In another embodiment, the liquid pharmaceutical composition is buffered to maintain a pH of about 6.0.

As noted above, the compositions of the present invention optionally may further comprise a pharmaceutically acceptable tonicity agent in addition to a chelating agent. As used herein, the terms “tonicity agent” or “tonicifier” refers to an excipient that can adjust the osmotic pressure of a liquid antibody formulation. In certain embodiments, the tonicity agent can adjust the osmotic pressure of a liquid antibody formulation to isotonic so that the antibody formulation is physiologically compatible with the cells of the body tissue of the subject. In still other embodiments, the “tonicity agent” may contribute to an improvement in stability of any of the anti-M-CSF antibodies described herein. An “isotonic” formulation is one that has essentially the same osmotic pressure as human blood. Isotonic formulations generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood. Isotonicity can be measured using a vapor pressure or ice-freezing type osmometer, for example.

The tonicity agent used to prepare the compositions of the present invention can exist in different forms. When the tonicity agent is referred to, it is intended that all of these different forms are encompassed by the name of the tonicity agent. For example, the tonicity agent can be in an enantiomeric (e.g., L- or D-enantiomer) or racemic form; isomers such as alpha or beta, including alpha, alpha; or beta, beta; or alpha, beta; or beta, alpha; a free acid or free base form; a hydrated form (e.g., monohydrate), or an anhydrous form.

In one embodiment, the tonicity agent is a saccharide. As used herein, the term “saccharide” refers to a class of molecules that are derivatives of polyhydric alcohols.

Saccharides are commonly referred to as carbohydrates and may contain different amounts of sugar (saccharide) units, e.g., monosaccharides, disaccharides and polysaccharides. Saccharides that are suitable for use as a tonicity agent in the present invention, include, but are not limited to, saccharides selected from the group consisting of fructose, glucose, mannose, sorbose, xylose, lactose, maltose, sucrose, dextran, pullulan, dextrin, cyclodextrins, soluble starch, hydroxyethyl starch, water-soluble glucans, and mixtures thereof.

In another embodiment, the tonicity agent is a polyol. As used herein, the term “polyol” refers an excipient with multiple hydroxyl groups, and includes sugars (reducing and nonreducing sugars), sugar alcohols and sugar acids. In one embodiment, the polyol has a molecular weight that is less than about 600 kD (e.g., in the range from about 120 to about 400 kD). A “reducing sugar” is one which contains a hemiacetal group that can reduce metal ions or react covalently with lysine and other amino groups in proteins and a “nonreducing sugar” is one which does not have these properties of a reducing sugar. Polyols that are suitable for use as a tonicity agent in the present invention, include, but are not limited to, polyols selected from the group consisting of mannitol, trehalose, sorbitol, erythritol, isomalt, lactitol, maltitol, xylitol, glycerol, lactitol, propylene glycol, polyethylene glycol, inositol, and mixtures thereof. In one embodiment, the tonicity agent is a non-reducing sugar selected from the group consisting of trehalose, sucrose, and mixtures thereof.

In one embodiment, the tonicity agent is mannitol. In another embodiment, the tonicity agent is D-mannitol. In another embodiment, the tonicity agent is trehalose. In another embodiment, the tonicity agent is α α-trehalose dihydrate. In another embodiment, the tonicity agent is sucrose.

In one embodiment, concentration of the tonicity agent in the liquid pharmaceutical composition ranges from about 1 millimolar to about 600 millimolar, from about 1 millimolar to about 400 millimolar, from 1 millimolar to about 300 millimolar, or from 200 millimolar to about 275 millimolar. In one another embodiment, the tonicity agent is mannitol and is present in the liquid pharmaceutical composition at a concentration of about 247 millimolar. In another embodiment, the tonicity agent is trehalose and is present in the liquid pharmaceutical composition at a concentration of about 222 millimolar. In another embodiment, the tonicity agent is trehalose and is present in the liquid pharmaceutical composition at a concentration of about 238 millimolar. In another embodiment, the tonicity agent is sucrose is present in the liquid pharmaceutical composition at a concentration of about 263 millimolar.

In one embodiment, concentration of the tonicity agent in the liquid pharmaceutical composition ranges from about 1 mg/ml to about 300 mg/ml, from about 1 mg/ml to about 200 mg/ml, or from about 50 mg/ml to about 150 mg/ml. In another embodiment, the tonicity agent is mannitol and is present in the liquid pharmaceutical composition at a concentration of about 45 mg/ml millimolar. In another embodiment, the tonicity agent is trehalose and is present in the liquid pharmaceutical composition at a concentration of about 84 mg/ml. In another embodiment, the tonicity agent is trehalose and is present in the liquid pharmaceutical composition at a concentration of about 90 mg/ml. In another embodiment, the tonicity agent is sucrose and is present in the liquid pharmaceutical composition at a concentration of about 90 mg/ml.

In one embodiment, the tonicity agent is a salt, such as sodium chloride. In one embodiment, when the tonicity agent is a salt, the concentration of the salt in the liquid pharmaceutical composition ranges from about 1 mg/ml to about 20 mg/ml. In another embodiment, the tonicity agent is sodium chloride and the concentration of the sodium chloride in the liquid pharmaceutical composition is about 8.18 mg/ml.

Ranges intermediate to the above-recited tonicity agent concentrations are also intended to be part of this invention. For example, ranges of values using a combination of any of the above-recited values as upper and/or lower limits are intended to be included.

As noted above, the compositions of the present invention optionally may further comprise a pharmaceutically acceptable surfactant in addition to a chelating agent. As used herein, the term “surfactant” refers to an excipient that can alter the surface tension of a liquid antibody formulation. In certain embodiments, the surfactant reduces the surface tension of a liquid antibody formulation. In still other embodiments, the “surfactant” may contribute to an improvement in stability of any of the anti-M-CSF antibodies described herein. For example, the surfactant may reduce aggregation of the formulated antibody and/or minimize the formation of particulates in the formulation and/or reduces adsorption. The surfactant may also improve stability of the antibody during and after a freeze/thaw cycle.

Suitable surfactants include polysorbate surfactants, poloxamers (e.g., poloxamer 18 and 407), triton surfactants such as Triton X-100®, polysorbate surfactants such as Tween 20° and Tween 80®, sodium dodecyl sulfate, sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyl-taurate, disodium methyl oleyl-taurate, dihydroxypropyl PEG 5 linoleammonium chloride, polyethylene glycol, polypropylene glycol, and mixtures thereof.

In one embodiment, the surfactant is a polysorbate surfactant comprising at least one excipient that is selected from the group consisting of polysorbate 20, polysorbate 21, polysorbate 40, polysorbate 60, polysorbate 61, polysorbate 65, polysorbate 80, polysorbate 81, polysorbate 85, and mixtures thereof. In another embodiment, the liquid pharmaceutical composition comprises polysorbate 80.

The concentration of the surfactant when present in the liquid pharmaceutical composition generally ranges from about 0.01 mg/ml to about 10 mg/ml, from about 0.05 mg/ml to about 5.0 mg/ml, from about 0.1 mg/ml to about 1.0 mg/ml, or from about 0.2 mg/ml to about 0.7 mg/ml. In another embodiment, the concentration of the surfactant ranges from about 0.05 millimolar to about 1.0 millimolar. In another embodiment, the surfactant is present in an amount that is about 0.2 mg/ml. In another embodiment, the surfactant is present in an amount that is about 0.5 mg/ml. In one embodiment, the liquid pharmaceutical composition contains about 0.2 mg/ml polysorbate 80. In another embodiment, the liquid pharmaceutical composition contains about 0.4 mg/ml polysorbate 80. In another embodiment, the liquid pharmaceutical composition contains about 0.5 mg/ml polysorbate 80.

Ranges intermediate to the above-recited surfactant concentrations are also intended to be part of this invention. For example, ranges of values using a combination of any of the above-recited values as upper and/or lower limits are intended to be included.

As noted above, the compositions of the present invention optionally may further comprise a pharmaceutically acceptable antioxidant in addition to a chelating agent. Suitable antioxidants include, but are not limited to, methionine, sodium thiosulfate, catalase, and platinum. For example, the liquid pharmaceutical composition may contain methionine in a concentration that ranges from 1 mM to about 100 mM, and in particular, is about 27 mM.

In one embodiment, the present invention encompasses a composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4, wherein the antibody binds to human M-CSF and the composition is substantially free of endotoxin.

In one embodiment, the present invention encompasses a composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4, wherein the antibody binds to human M-CSF and the composition has a concentration of endotoxin of from about 0.001 to about 1 endotoxin units per milligram of antibody (EU/mg).

In one embodiment, the present invention encompasses a composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4, wherein the antibody binds to human M-CSF and the composition has a concentration of endotoxin of from about 0.001 to about 0.5 endotoxin units per milligram of antibody (EU/mg).

In one embodiment, the present invention encompasses a composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4, wherein the antibody binds to human M-CSF and the composition has a concentration of endotoxin of from about 0.001 to about 0.2 endotoxin units per milligram of antibody (EU/mg).

In one embodiment, the present invention encompasses a composition comprising at least one human monoclonal IgG2 anti-M-CSF antibody having the heavy and light chain amino acid sequences of antibody 8.10.3F, wherein the antibody binds to human M-CSF and the composition has a concentration of endotoxin of from about 0.001 to about 1.0 endotoxin units per milligram of antibody (EU/mg).

In one embodiment, the present invention encompasses a composition comprising at least one human monoclonal IgG2 anti-M-CSF antibody having the heavy and light chain amino acid sequences of antibody 8.10.3F, wherein the antibody binds to human M-CSF and the composition has a concentration of endotoxin of from about 0.001 to about 0.5 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4, wherein the antibody binds to human M-CSF; and a pharmaceutically acceptable excipient, wherein the composition is substantially free of endotoxin.

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4, wherein the antibody binds to human M-CSF; and a chelating agent, wherein the composition is substantially free of endotoxin.

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4, wherein the antibody binds to human M-CSF and has a purity of at least about 95%; and a chelating agent, wherein the composition is substantially free of endotoxin.

In another embodiment, the invention is directed to a composition comprising an anti-M-CSF antibody and a chelating agent, wherein the composition is substantially free of endotoxin. In another embodiment, the invention is directed to a composition comprising an anti-M-CSF antibody and EDTA, wherein the composition is substantially free of endotoxin. In another embodiment, the invention is directed to a composition comprising an anti-M-CSF antibody and DTPA, wherein the composition is substantially free of endotoxin. In another embodiment, the invention is directed to a composition comprising an anti-M-CSF antibody, a chelating agent, and a buffer, wherein the composition is substantially free of endotoxin. In another embodiment, the invention is directed to a composition comprising an anti-M-CSF antibody, a chelating agent, and histidine, wherein the composition is substantially free of endotoxin. In another embodiment, the invention is directed to a composition comprising an anti-M-CSF antibody, EDTA, and histidine, wherein the composition is substantially free of endotoxin. In another embodiment, the invention is directed to a composition comprising an anti-M-CSF antibody, histidine, polysorbate 80, EDTA and sucrose, wherein the composition is substantially free of endotoxin.

In another embodiment, the invention is directed to a composition comprising an anti-M-CSF antibody, a chelating agent, and a tonicity agent, wherein the composition is substantially free of endotoxin. In another embodiment, the invention is directed to a composition comprising an anti-M-CSF antibody, a chelating agent, and mannitol, wherein the composition is substantially free of endotoxin. In another embodiment, the invention is directed to a composition comprising an anti-M-CSF antibody, a chelating agent, and trehalose, wherein the composition is substantially free of endotoxin. In another embodiment, the invention is directed to a composition comprising an anti-M-CSF antibody, EDTA, and trehalose, wherein the composition is substantially free of endotoxin. In another embodiment, the invention is directed to a composition comprising an anti-M-CSF antibody, EDTA, and mannitol, wherein the composition is substantially free of endotoxin. In another embodiment, the invention is directed to a composition comprising an anti-M-CSF antibody, EDTA, and sucrose, wherein the composition is substantially free of endotoxin. In another embodiment, the invention is directed to a composition comprising an anti-M-CSF antibody, DTPA, and trehalose, wherein the composition is substantially free of endotoxin. In another embodiment, the invention is directed to a composition comprising an anti-M-CSF antibody, DTPA, and mannitol, wherein the composition is substantially free of endotoxin.

In another embodiment, the invention is directed to a composition comprising an anti-M-CSF antibody, a chelating agent, and a surfactant, wherein the composition is substantially free of endotoxin. In another embodiment, the invention is directed to a composition comprising an anti-M-CSF antibody, EDTA, and a surfactant, wherein the composition is substantially free of endotoxin. In another embodiment, the invention is directed to a composition comprising an anti-M-CSF antibody, DTPA, and a surfactant, wherein the composition is substantially free of endotoxin. In another embodiment, the invention is directed to a composition comprising an anti-M-CSF antibody, a chelating agent selected from the group consisting of EDTA and DTPA, and polysorbate 80, wherein the composition is substantially free of endotoxin.

In another embodiment, the invention is directed to a composition comprising anti-M-CSF antibody, a buffer, and a surfactant, wherein the composition is substantially free of endotoxin. In another embodiment, the invention is directed to a composition comprising anti-M-CSF antibody, histidine, and a surfactant, wherein the composition is substantially free of endotoxin. In another embodiment, the invention is directed to a composition comprising anti-M-CSF antibody, histidine, and polysorbate 80, wherein the composition is substantially free of endotoxin.

In another embodiment, the invention is directed to a composition comprising an anti-M-CSF antibody, a chelating agent, a buffer, and a surfactant, wherein the composition is substantially free of endotoxin. In another embodiment, the invention is directed to a composition comprising an anti-M-CSF antibody, a chelating agent, a buffer, and a tonicity agent.

In another embodiment, the invention is directed to a composition comprising an anti-M-CSF antibody, a chelating agent, a buffer, a surfactant, and a tonicity agent, wherein the composition is substantially free of endotoxin. In another embodiment, the invention is directed to composition comprising an anti-M-CSF antibody and histidine, wherein the composition is substantially free of endotoxin.

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; and a chelating agent, wherein the antibody binds to human M-CSF, and the composition has an antibody concentration of from about 1.0 mg/ml to about 100 mg/ml and a concentration of endotoxin of from about 0.001 to about 1.0 endotoxin units per milligram of antibody (EU/mg).

In one embodiment, the present invention encompasses a liquid pharmaceutical composition comprising at least one human monoclonal IgG2 anti-M-CSF antibody having the heavy and light chain amino acid sequences of antibody 8.10.3F; and a chelating agent, wherein the antibody binds to human M-CSF, and the composition has an antibody concentration of from about 1.0 mg/ml to about 100 mg/ml and a concentration of endotoxin of from about 0.001 to about 1.0 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; and a chelating agent, wherein the antibody binds to human M-CSF, and the composition contains a concentration of antibody that is at least about 5 mg/ml, at least about 10 mg/ml, at least about 15 mg/ml or at least about 20 mg/ml and has a concentration of endotoxin of from about 0.001 to about 1.0 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; and a chelating agent, wherein the antibody binds to human M-CSF, and the composition contains a concentration of antibody that is about 10 mg/ml and has a concentration of endotoxin of from about 0.001 to about 1.0 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; and a chelating agent, wherein the antibody binds to human M-CSF, and the composition contains a concentration of antibody that is about 20 mg/ml and has a concentration of endotoxin of from about 0.001 to about 1.0 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; and from about 0.01 millimolar to about 0.5 millimolar of chelating agent, wherein the antibody binds to human M-CSF, and the composition has an antibody concentration of from about 1.0 mg/ml to about 100 mg/ml and a concentration of endotoxin of from about 0.001 to about 1.0 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; and from about 0.01 millimolar to about 0.5 millimolar of chelating agent, wherein the antibody binds to human M-CSF, and the composition has an antibody concentration of from about 1.0 mg/ml to about 100 mg/ml and a concentration of endotoxin of from about 0.001 to about 0.5 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; and from about 0.01 millimolar to about 0.5 millimolar of chelating agent, wherein the antibody binds to human M-CSF, and the composition has an antibody concentration of from about 1.0 mg/ml to about 100 mg/ml and a concentration of endotoxin of from about 0.001 to about 0.2 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; and about 0.05 millimolar of chelating agent, wherein the antibody binds to human M-CSF, and the composition has an antibody concentration of from about 1.0 mg/ml to about 100 mg/ml and a concentration of endotoxin of from about 0.001 to about 1.0 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; and from about 0.01 millimolar to about 0.5 millimolar of EDTA, wherein the antibody binds to human M-CSF, and the composition has an antibody concentration of from about 1.0 mg/ml to about 100 mg/ml and a concentration of endotoxin of from about 0.001 to about 1.0 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; and from about 1.0 millimolar to about 100 millimolar of histidine, wherein the antibody binds to human M-CSF, and the composition has an antibody concentration of from about 1.0 mg/ml to about 100 mg/ml and a concentration of endotoxin of from about 0.001 to about 1.0 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; from about 0.01 millimolar to about 0.5 millimolar of EDTA; and from about 1 millimolar to about 50 millimolar of histidine, wherein the antibody binds to human M-CSF, and the composition has an antibody concentration of from about 1.0 mg/ml to about 100 mg/ml and a concentration of endotoxin of from about 0.001 to about 1.0 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; from about 0.01 millimolar to about 0.5 millimolar of EDTA; from about 0.01 millimolar to about 0.5 millimolar of EDTA; from about 1 millimolar to about 50 millimolar of histidine; and from about 200 millimolar to about 300 millimolar of mannitol, wherein the antibody binds to human M-CSF, and the composition has an antibody concentration of from about 1.0 mg/ml to about 100 mg/ml and a concentration of endotoxin of from about 0.001 to about 1.0 endotoxin units per milligram of antibody (EU/mg).

In one embodiment, the present invention encompasses a liquid pharmaceutical composition comprising at least one antibody comprising an amino acid sequence that is at least 95% identical to a heavy chain amino acid sequence shown in SEQ ID NO: 2, and further comprising an amino acid sequence that is at least 95% identical to a light chain amino acid sequence shown in SEQ ID NO: 4; from about 0.01 millimolar to about 0.5 millimolar of EDTA; from about 0.01 millimolar to about 0.5 millimolar of EDTA; from about 1 millimolar to about 50 millimolar of histidine; and from about 200 millimolar to about 300 millimolar of trehalose, wherein the antibody binds to human M-CSF, and the composition has an antibody concentration of from about 1.0 mg/ml to about 100 mg/ml and a concentration of endotoxin of from about 0.001 to about 1.0 endotoxin units per milligram of antibody (EU/mg).

In another embodiment, the invention is directed to a stable liquid pharmaceutical composition comprising an anti-M-CSF antibody and a pharmaceutically acceptable chelating agent, wherein the molar concentration of the antibody ranges from about 0.0006 millimolar to about 1.35 millimolar and the molar concentration of the chelating agent ranges from about 0.003 millimolar to about 50 millimolar, and wherein the molar ratio of antibody to chelating agent ranges from about 0.00001 to about 450; from about 0.0001 to about 100; from about 0.005 to about 50; from about 0.001 to about 10; from about 0.01 to about 5; from about 0.1 to about 1; or is about 0.5; and wherein the composition has a concentration of endotoxin of from about 0.001 to about 1.0 endotoxin units per milligram of antibody (EU/mg).

### Routes of Administration and Dosages:

The compositions of this invention may be in liquid solutions (e.g., injectable and infusible solutions). The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, intramuscular, and intrasternally) or by infusion techniques, in the form of sterile injectable aqueous or olagenous suspensions. As will be appreciated by the skilled artisan, the route and/or mode of administration will vary depending upon the desired results. In a preferred embodiment, the antibody is administered by intravenous infusion or injection. In another preferred embodiment, the antibody is administered by intramuscular or subcutaneous injection. Therapeutic compositions typically are sterile and stable under the conditions of manufacture and storage.

The composition can be formulated as a solution, microemulsion, dispersion, or liposome. Sterile injectable solutions can be prepared by incorporating the anti-M-CSF antibody in the required amount in an appropriate diluent with one or a combination of ingredients enumerated above, as required, followed by sterilization (e.g., filter sterilization). Generally, dispersions are prepared by incorporating the active compound into a sterile vehicle that contains a basic dispersion medium and the required other ingredients from those enumerated above. Such suspensions may be formulated according to the known art using those suitable dispersing of wetting agents and suspending agents or other acceptable agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, n-3 polyunsaturated fatty acids may find use in the preparation of injectables.

In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants.

Prolonged absorption of injectable compositions can be brought about by including in the composition an agent that delays absorption, for example, monostearate salts and gelatin or by formulating the composition into prolonged absorption forms such as, depots, liposomes, polymeric microspheres, polymeric gels, and implants.

Other methods for administration of the antibodies described herein include dermal patches that release the medications directly into a subject's skin. Such patches can contain the antibodies of the present invention in an optionally buffered, liquid solution, dissolved and/or dispersed in an adhesive, or dispersed in a polymer.

Still other methods for administration of the antibodies described herein include liquid opthalmological drops for the eyes.

The antibody may be administered once, but more preferably is administered multiple times. For example, the antibody may be administered from once daily to once every six months or longer. The administering may be on a schedule such as three times daily, twice daily, once daily, once every two days, once every three days, once weekly, once every two weeks, once every month, once every two months, once every three months and once every six months.

The antibody may also be administered continuously via a minipump. The antibody may be administered at the site of a tumor or inflamed body part, into the tumor or inflamed body part or at a site distant from the site of the tumor or inflamed body part. The antibody may be administered once, at least twice or for at least the period of time until the condition is treated, palliated or cured. The antibody generally may be administered for as long as the tumor or inflammation is present provided that the antibody causes the tumor or cancer to stop growing or to decrease in weight or volume or until the inflamed body part experiences a reduction in inflammation. The antibody typically would be administered as part of a pharmaceutical composition as described supra.

The compositions of the invention may include a therapeutically effective amount or a prophylactically effective amount of an antibody or antigen-binding portion of the invention. In preparing the composition, the therapeutically effective amount of the anti-M-CSF antibody present in the composition can be determined, for example, by taking into account the desired dose volumes and mode(s) of administration, the nature and severity of the condition to be treated, and the age and size of the subject.

Exemplary, non-limiting dose ranges for administration of the pharmaceutical compositions of the present invention to a subject are from about 0.01 mg/kg to about 200 mg/kg (expressed in terms of milligrams (mg) of anti-M-CSF antibody administered per kilogram (kg) of subject weight), from about 0.01 mg/kg to about 100 mg/kg, from about 0.01 mg/kg to about 10 mg/kg, from about 0.1 mg/kg to about 10 mg/kg, or from about 0.1 mg/kg to about 3 mg/kg For purposes of the present invention, an average human subject weighs about 70 kg. In addition, the quantity of active component in a unit dose preparation may be varied or adjusted from 0.1 mg to 100 mg and from 0.5 mg to 100 mg, according to the particular application and the potency of the active component. Ranges intermediate to any of the dosages cited herein, e.g., about 0.01 mg/kg-199 mg/kg, are also intended to be part of this invention. For example, ranges of values using a combination of any of the recited values as upper and/or lower limits are intended to be included.

Dosage regimens can also be adjusted to provide the optimum desired response (e.g., a therapeutic or prophylactic response) by administering several divided doses to a subject over time or the dose can be proportionally reduced or increased as indicated by the exigencies of the therapeutic situation. It is especially advantageous to formulate parenteral compositions in dosage unit form for ease of administration and uniformity of dosage.

Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the anti-M-CSF antibody or portion and the particular therapeutic or prophylactic effect to be achieved, and (b) the limitations inherent in the art of compounding such an antibody for the treatment of sensitivity in individuals.

The liquid formulations of the present invention can be prepared as unit dosage forms. For example, a unit dosage per vial may contain from 1 to 1000 milliliters (mls) of different concentrations of an anti-M-CSF antibody. In other embodiments, a unit dosage per vial may contain about 1 ml, 2 ml, 3 ml, 4 ml, 5 ml, 6 ml, 7 ml, 8 ml, 9 ml, 10 ml, 15 ml, 20 ml, 30 ml, 40 ml, 50 ml or 100 ml of different concentrations of an anti-M-CSF antibody. If necessary, these preparations can be adjusted to a desired concentration by adding a sterile diluent to each vial.

The liquid formulations of the present invention can also be prepared as unit dosage forms in sterile bags or containers, which are suitable for connection to an intravenous administration line or catheter.

### Methods of Treatment:

Any of the types of antibodies described herein may be used therapeutically. In a preferred embodiment, the anti-M-CSF antibody is a human antibody. In another preferred embodiment, the M-CSF is human M-CSF and the subject is a human subject. In yet another preferred embodiment, the anti-M-CSF antibody is a human IgG2 antibody. Alternatively, the subject may be a mammal that expresses an M-CSF protein that the anti-M-CSF antibody cross-reacts with. The antibody may be administered to a non-human mammal expressing M-CSF with which the antibody cross-reacts (i.e., a primate) for veterinary purposes or as an animal model of human disease. Such animal models may be useful for evaluating therapeutic efficacy of antibodies of this invention.

In one embodiment, the present invention provides a method for the treatment of a M-CSF-mediated disorder in a subject, comprising administering to the subject a therapeutically effective amount of a liquid pharmaceutical composition comprising: at least one antibody comprising an amino acid sequence that is at least 90% identical to a light chain amino acid sequence shown in SEQ ID NO: 4, and further comprising an amino acid sequence that is at least 90% identical to a heavy chain amino acid sequence shown in SEQ ID NO: 2, wherein the antibody binds to human M-CSF and the composition is substantially free of endotoxin; and a pharmaceutically acceptable excipient.

In one embodiment, the present invention provides a method for the treatment of an inflammatory disease in a subject, comprising administering to the subject a therapeutically effective amount of a liquid pharmaceutical composition comprising: at least one antibody comprising an amino acid sequence that is at least 90% identical to a light chain amino acid sequence shown in SEQ ID NO: 4, and further comprising an amino acid sequence that is at least 90% identical to a heavy chain amino acid sequence shown in SEQ ID NO: 2, wherein the antibody binds to human M-CSF and the composition is substantially free of endotoxin; and a pharmaceutically acceptable excipient comprising a chelating agent alone or in combination with other excipients chosen from a buffer, antioxidant, a tonicity agent, or a surfactant, and mixtures thereof. In further embodiments, the aforementioned subject is one that is in need of the treatment of an inflammatory disease. In other embodiments, the methods and compositions of the present invention encompass the treatment of the inflammatory diseases selected from the group consisting of atherosclerosis, sepsis, asthma, autoimmune diseases, osteoporosis, rheumatoid arthritis, and osteoarthritis.

In another embodiment, the present invention provides a method for the treatment of a neoplasia disorder in a subject, comprising administering to the subject a therapeutically effective amount of a liquid pharmaceutical composition comprising: at least one antibody comprising an amino acid sequence that is at least 90% identical to a light chain amino acid sequence shown in SEQ ID NO: 4, and further comprising an amino acid sequence that is at least 90% identical to a heavy chain amino acid sequence shown in SEQ ID NO: 2, wherein the antibody binds to human M-CSF and the composition is substantially free of endotoxin; and a pharmaceutically acceptable excipient comprising a chelating agent alone or in combination with other excipients chosen from a buffer, an antioxidant, a tonicity agent, or a surfactant, and mixtures thereof. In further embodiments, the aforementioned subject is one that is in need of the treatment of a neoplasia disorder.

Both of the terms, “neoplasia” and “neoplasia disorder”, refer to a “neoplasm” or tumor, which may be benign, premalignant, metastatic, or malignant. Also encompassed by the present invention are benign, premalignant, metastatic, or malignant neoplasias. Also encompassed by the present invention are benign, premalignant, metastatic, or malignant tumors. Thus, all of benign, premalignant, metastatic, or malignant neoplasia or tumors are encompassed by the present invention and may be referred to interchangeably, as neoplasia, neoplasms or neoplasia-related conditions. Tumors are generally known in the art to be a mass of neoplasia or “neoplastic” cells. Although, it is to be understood that even one neoplastic cell is considered, for purposes of the present invention to be a neoplasm or alternatively, neoplasia.

Neoplasia disorders that may be treated by an anti-M-CSF antibody of the invention can involve any tissue or organ, and include, but are not limited to bone, brain, lung, squamous cell, bladder, gastric, pancreatic, breast, head, neck, liver, renal, ovarian, prostate, colorectal, esophageal, gynecological (e.g., cervical and ovarian), nasopharynx, or thyroid cancers. Also encompassed by the term neoplasia disorders, are bone metastases, melanomas, lymphomas, leukemias, and multiple myelomas. In particular, the anti-M-CSF antibody formulations of the present invention are useful to treat cancers of the breast, prostate, colon and lung.

In other embodiments, the methods and compositions of the present invention encompass the prevention and treatment of the neoplasia disorders selected from the group consisting of acral lentiginous melanoma, actinic keratoses, adenocarcinoma, adenoid cycstic carcinoma, adenomas, familial adenomatous polyposis, familial polyps, colon polyps, polyps, adenosarcoma, adenosquamous carcinoma, adrenocortical carcinoma, AIDS-related lymphoma, anal cancer, astrocytic tumors, bartholin gland carcinoma, basal cell carcinoma, bile duct cancer, bladder cancer, brain stem glioma, brain tumors, breast cancer, bronchial gland carcinomas, capillary carcinoma, carcinoids, carcinoma, carcinoma of the fallopian tubes, carcinoma of the endometrium, carcinosarcoma, cavernous, central nervous system lymphoma, cerebral astrocytoma, cholangiocarcinoma, chondosarcoma, choriod plexus papilloma/carcinoma, clear cell carcinoma, skin cancer, brain cancer, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, epitheloid, esophageal cancer, Ewing's sarcoma, extragonadal germ cell tumor, fibrolamellar, focal nodular hyperplasia, gallbladder cancer, gastrinoma, germ cell tumors, gestational trophoblastic tumor, glioblastoma, glioma, glucagonoma, hemangiblastomas, hemangioendothelioma, hemangiomas, hepatic adenoma, hepatic adenomatosis, hepatocellular carcinoma, Hodgkin's lymphoma, hypopharyngeal cancer, hypothalamic and visual pathway glioma, insulinoma, intaepithelial neoplasia, interepithelial squamous cell neoplasia, intraocular melanoma, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related conditions, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative conditions, nasal cavity and paranasal sinus cancer, nasopharyngeal cancer, neuroblastoma, neuroepithelial adenocarcinoma nodular melanoma, neoplasms of the central nervous system (e.g., primary CNS lymphoma, spinal axis tumors, brain stem gliomas or pituitary adenomas), non-Hodgkin's lymphoma, oat cell carcinoma, oligodendroglial, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, retinoblastoma, rhabdomyosarcoma, sarcoma, serous carcinoma, small cell carcinoma, small intestine cancer, soft tissue carcinomas, somatostatin-secreting tumor, squamous carcinoma, squamous cell carcinoma, submesothelial, superficial spreading melanoma, supratentorial primitive neuroectodermal tumors, thyroid cancer, undifferentiatied carcinoma, urethral cancer, uterine cancer, uveal melanoma, verrucous carcinoma, vaginal cancer, vipoma, vulvar cancer, Waldenstrom's macroglobulinemia, well differentiated carcinoma, and Wilm's tumor.

In a more preferred embodiment, the anti-M-CSF antibody is administered to a subject with breast cancer, prostate cancer, lung cancer or colon cancer. In an even more preferred embodiment, the method causes the cancer to stop proliferating abnormally, or not to increase in weight or volume or to decrease in weight or volume.

The compositions of the present invention may be used in combination with agents useful for treating a cancer in a mammal such as chemotherapeutic agents. In some embodiments, the chemotherapeutic agent is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, tamoxifen, anti-hormones, e.g., anti-androgens, and anti-angiogenesis agents.

In addition, a composition of a human anti-M-CSF monoclonal antibody of the invention can also be used with signal transduction inhibitors, such as agents that can inhibit EGF-R (epidermal growth factor receptor) responses, such as EGF-R antibodies, EGF antibodies, and molecules that are EGF-R inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, HERCEPTIN™ (Genentech, Inc.). EGFR-inhibiting agents include, but are not limited to, the monoclonal antibodies C225 and anti-EGFR 22Mab (ImClone Systems Incorporated), ABX-EGF (Abgenix/Cell Genesys), EMD-7200 (Merck KgaA), EMD-5590 (Merck KgaA), MDX-447/H-477 (Medarex Inc. and Merck KgaA), and the compounds ZD-1834, ZD-1838 and ZD-1839 (AstraZeneca), PKI-166 (Novartis), PKI-166/CGP-75166 (Novartis), PTK 787 (Novartis), CP 701 (Cephalon), leflunomide (Pharmacia/Sugen), CI-1033 (Warner Lambert Parke Davis), CI-1033/PD 183,805 (Warner Lambert Parke Davis), CL-387,785 (Wyeth-Ayerst), BBR-1611 (Boehringer Mannheim GmbH/Roche), Naamidine A (Bristol Myers Squibb), RC-3940-II (Pharmacia), BIBX-1382 (Boehringer Ingelheim), OLX-103 (Merck & Co.), VRCTC-310 (Ventech Research), EGF fusion toxin (Seragen Inc.), DAB-389 (Seragen/Lilgand), ZM-252808 (Imperial Cancer Research Fund), RG-50864 (INSERM), LFM-A12 (Parker Hughes Cancer Center), WHI-P97 (Parker Hughes Cancer Center), GW-282974 (Glaxo), KT-8391 (Kyowa Hakko) and EGF-R Vaccine (York Medical/Centro de Immunologia Molecular (CIM)). These and other EGF-R-inhibiting agents can be used in the present invention.

VEGF inhibitors, for example SU-5416 and SU-6668 (Sugen Inc.), AVASTIN™ (Genentech), SH-268 (Schering), and NX-1838 (NeXstar) can also be combined with the compound of the present invention. Anti-inflammatory agents can be used in conjunction with an anti-M-CSF antibody formulation of the present invention. For the treatment of inflammatory diseases such as rheumatoid arthritis, the human anti-M-CSF antibodies of the invention may be combined with agents such as TNF-α inhibitors such as TNF drugs (such as REMICADE™, CDP-870 and HUMIRA™) and TNF receptor immunoglobulin molecules (such as ENBREL™), CTLA-4Ig, anti-CD20 antibodies (e.g., rituxamab), IL-6 antibodies, IL-6 receptor antibodies (e.g., tocilizumab), IL-1 inhibitors, IL-1 receptor antagonists or soluble IL-1 ra (e.g. Kineret or ICE inhibitors), COX-2 inhibitors (such as celecoxib, rofecoxib, valdecoxib and etoricoxib), metalloprotease inhibitors (preferably MMP-13 selective inhibitors), p2X7 inhibitors, α2δ ligands (such as NEURONTIN™ AND PREGABALIN™), low dose methotrexate, sulfasalazine, Mesalamine ieflunomide, hydroxychloroquine, d-penicillamine, auranofin or parenteral or oral gold.

The compositions of the invention can also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flurbiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc.

The human anti-M-CSF antibody compositions of the present invention may also be used in combination with cardiovascular agents such as calcium channel blockers, lipid lowering agents such as statins (e.g., atorvastain calcium), fibrates, beta-blockers, ACE inhibitors, Angiotensin-2 receptor antagonists, and platelet aggregation inhibitors.

The compositions of the present invention may also be used in combination with CNS agents such as antidepressants (such as sertraline), anti-Parkinsonian drugs (such as deprenyl, L-dopa, REQUIP™, MIRAPEX™, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti-Alzheimer's drugs such as donepezil, tacrine, α2δ LIGANDS (such NEUROTIN™ and PREGABALIN™) inhibitors, COX-2 inhibitors, propentofylline or metrifonate.

The anti-M-CSF antibody compositions of the present invention may also be used in combination with osteoporosis agents such as roloxifene, droloxifene, lasofoxifene or fosomax and immunosuppressant agents such as FK-506 and rapamycin.

### Articles of Manufacture

In another embodiment of the invention, an article of manufacture is provided comprising a container, which holds the liquid pharmaceutical composition comprising at least one of the monoclonal anti-M-CSF antibodies of the present invention in combination with a pharmaceutically acceptable excipient that is substantially free of endotoxin, and optionally provides instructions for its use. Suitable containers include, for example, bottles, bags, vials and syringes. The container may be formed from a variety of materials such as glass or plastic. An exemplary container is a 3-20 cc single use glass vial. Alternatively, for a multidose formulation, the container may be 3-100 cc glass vial. The container holds the formulation and the label on, or associated with, the container may indicate directions for use. The article of manufacture may further include other materials desirable from a commercial and user standpoint, including other buffers, diluents, filters, needles, syringes, and package inserts with instructions for use, contraindications, and/or lists of potential side-effects.

The present invention also provides a kit for preparing a liquid composition of an antibody comprising a first container comprising monoclonal anti-M-CSF antibody 8.10.3F, which is substantially free of endotoxin and a second container comprising a pharmaceutically acceptable excipient.

The following examples describe embodiments of the invention. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples. In the examples, all percentages are given on a weight basis unless otherwise indicated. The skilled artisan will appreciate that the weight quantities and/or weight-to-volume ratios recited in the examples can be converted to moles and/or molarities using the art-recognized molecular weights of the recited ingredients. Weight quantities exemplified herein (e.g., grams) are for the volumes (e.g., of buffer solutions, antibody formulation, etc.) recited. The skilled artisan will appreciate that the weight quantities can be proportionally adjusted when different formulation volumes are desired.

### Example 1

This Example shows the generation of hybridoma cell lines that produce anti-M-CSF antibodies as described in U.S. Published Application No. 20050059113 to Bedian, et al.

### Immunization and Hybridoma Generation

Eight to ten week old XENOMOUSE™ mice were immunized intraperitoneally or in their hind footpads with human M-CSF (10 pg/dose/mouse). This dose was repeated five to seven times over a three to eight week period. Four days before fusion, the mice were given a final injection of human M-CSF in phosphate buffered saline (PBS). The spleen and lymph node lymphocytes from immunized mice were fused with the non-secretory myeloma P3-X63-Ag8.653 cell line, and the fused cells were subjected to HAT selection. See Galfre, G. and Milstein, C., “Preparation of monoclonal antibodies: strategies and procedures.” Methods Enzymol. 73:3-46 (1981). A panel of hybridomas all secreting M-CSF specific human IgG2 and IgG4 antibodies was recovered. Antibodies also were generated using XENOMAX™ technology as described in Babcook, J. S. et al., Proc. Natl. Acad. Sci. USA 93:7843-48, 1996. Nine cell lines engineered to produce antibodies of the invention were selected for further study and designated 252, 88, 100, 3.8.3, 2.7.3, 1.120.1, 9.14.4, 8.10.3 and 9.7.2. The hybridomas were deposited under terms in accordance with the Budapest Treaty with the American Type Culture Collection (ATCC), 10801 University Blvd., Manassas, Va. 20110-2209 on Aug. 8, 2003. The hybridomas were assigned the following accession numbers:

| | |
|---|---|
| Hybridoma 3.8.3 (LN 15891) | PTA-5390 |
| Hybridoma 2.7.3 (LN 15892) | PTA-5391 |
| Hybridoma 1.120.1 (LN 15893) | PTA-5392 |
| Hybridoma 9.7.2 (LN 15894) | PTA-5393 |
| Hybridoma 9.14.4 (LN 15895) | PTA-5394 |
| Hybridoma 8.10.3 (LN 15896) | PTA-5395 |
| Hybridoma 88-gamma (UC 25489) | PTA-5396 |
| Hybridoma 88-kappa (UC 25490) | PTA-5397 |
| Hybridoma 100-gamma (UC 25491) | PTA-5398 |
| Hybridoma 100-kappa (UC 25492) | PTA-5399 |
| Hybridoma 252-gamma (UC 25493) | PTA-5400 |
| Hybridoma 252-kappa (UC 25494) | PTA-5401 |

### Example 2

This Example shows the generation of a recombinant mammalian cell line that produces anti-M-CSF antibodies.

DNA encoding the heavy and light chains of monoclonal antibodies 8.10.3 was cloned from the respective hybridoma cell line 8.10.3 and the DNA sequences were determined by methods known to one skilled in the art. The DNA from the hybridoma cell line 8.10.3 was mutated at specific framework regions in the variable domain to obtain 8.10.3F. From nucleic acid sequence and predicted amino acid sequence of the antibody 8.10.3F, the identity of the gene usage for each antibody chain was determined by (“VBASE”). Table 2 sets forth the gene utilization of antibody 8.10.3F in accordance with the present invention:

TABLE 2
Heavy and Light Chain Human Gene Utilization and Sequences

| | Heavy Chain | | | | Light Chain | | |
|---|---|---|---|---|---|---|---|
| | SEQ ID | | | | SEQ ID | | |
| Antibody | NO: | V_{H} | D_{H} | J_{H} | NO: | V_{K} | J_{K} |
| 8.10.3F | 1 (nucleic | 3-48 | 1-26 | 4b | 3 (nucleic | A27 | 4 |
| | acid) | | | | acid) | | |
| | 2 (amino | | | | 4 (amino | | |
| | acid) | | | | acid) | | |

Antibody 8.10.3F DNA sequence inserts were obtained from the hybridoma cell line and subcloned into expression vectors. The expression vectors were then transfected into a mouse myeloma (NS0) host cell line to generate a primary transfectant cell line producing anti-M-CSF antibodies having the heavy and light chain sequences of 8.10.3F. Finally, samples of the 8.10.3F antibody producing NSO cell line were frozen and stored in liquid nitrogen.

### Example 3

This Example shows the production of anti-M-CSF 8.10.3F antibodies from the NS0 cell line generated according to Example 2.

A vial of 8.10.3F subcloned NS0 cells was removed from liquid nitrogen storage as described in Example 2. The frozen cells were thawed rapidly to 37° C. until the last ice crystal disappeared. The entire contents (1 milliliter) of the thawed vial were then pipetted into a 75 cm² T-Flask. Fourteen milliliters of prewarmed (36.5° C.±1.0° C.) CD Hybridoma growth medium (available from Invitrogen, Carlsbad, Calif.) containing 10% Low IgG containing fetal bovine serum (available from Invitrogen, Carlsbad, Calif.) was slowly pipetted into the T-Flask.

The flask was planted at a target viable cell density from about 2.0×10⁵ to about 5.0×10⁵ cells/ml. The flask was then placed in an incubator having a carbon dioxide level of 9% and a temperature of 36.5° C. and the cells were grown for about 3 days. At the end of this period, targeted cell number was on the order of 1.0 to 3.0×10⁶ cells/ml.

After the cells were grown for about 3 days, they were split so that a target cell density of 2.5×10⁵+/−0.5×10⁵ was achieved and then disposable shake flasks (i.e., seed flasks) were seeded based on cell density. Each shake flask contained CD Hybridoma growth media containing 10% Low IgG containing fetal bovine serum, with a final volume of cells and medium being 25 milliliters. The flasks were then shaken at 100+/−10 rpm at 36.5° C.±1.0° C. for about 3 days. Cell density in each flask at the end of this period was 1.0 to 3.0×10⁶ cells/ml and greater than 80% of the cells were viable.

After the cells were grown for about 3 days, the broth was harvested. Clarified broth was obtained after centrifugation for 15 minutes at 7000 rpm and subsequent filtration with a sterile 0.22 μm 4 inch Opticap™ Millipore™ filter into a sterile TC-Tech™ bag.

### Example 4

This Example shows a process for reducing the endotoxin content of a clarified broth containing anti-M-CSF 8.10.3F antibodies prepared according to Example 3. The values for the below purifications where a range of endotoxin level is expressed, were all determined using the gel clot assay (See Example 8). Where the endotoxin levels are expressed as a single measurement, the endotoxin level was determined by the Cambrex Kinetic-Quantitative Chromogenic LAL assay (See Example 7).

rProtein A Chromatography

A clarified broth prepared according to Example 3 was loaded directly onto a 150×50 mm column packed with rProtein A Sepharose® FF resin (Amersham, Piscataway, N.J.) equilibrated with an equilibration solution containing 50 mM sodium phosphate and 250 mM sodium chloride at pH 7.0. A resin bed height of 15 cm was used. Loading, washing, and elution for the column used a linear flow rate of 150 cm per hour.

Once the clarified broth was loaded onto the column (maximal load of 25 mg/ml of resin), the column was washed with 5 column volumes of a first wash solution containing 50 mM sodium phosphate (mixture of mono and dibasic sodium phosphate) and 250 mM sodium chloride at pH 7.0, followed by 5 column volumes of a second wash solution containing 25 mM sodium acetate at pH 5.5.

The column was then eluted with 4 column volumes of an elution buffer containing 25 mM sodium acetate at pH 3.5. After the clarified broth was passed through the rProtein A column, the endotoxin content was measured by gel clot LAL assay to be between 4.1 to 10.2 EU/mg of anti-M-CSF antibody. The eluent was then diluted 1:1 with a solution containing 50 mM Tris and 25 mM sodium chloride at pH 8.0. The pH of the diluted eluent was then adjusted to 8.0 with 1.5 M Tris base and the conductivity was adjusted to be less than 6 mS/cm with sterile water.

### Anion Exchange Chromatography

The rProtein A column eluent was then loaded onto a Q Sepharose® FF column (Amersham, Piscataway, N.J.). The Q Sepharose® column is an ion exchange chromatography column, and in particular, an anion exchange column containing a quaternary ammonium group. As above, a 15 cm bed height was used and the column diameter was varied from 1 to 5 cm (depending on the material load). Before the column was used, it was equilibrated with 6 column volumes of a solution containing 50 mM Tris and 25 mM sodium chloride at pH 8.0. Next, material from the rProtein A column elution was loaded directly onto the anion exchange column at a flow rate of 150 cm/hr. The typical load maximum used for the column was 20 mg/ml of resin. The pass through (non-bound fraction) contained the material of interest, and once all of the material from the rProtein A column was loaded, the column was washed with 4 column volumes of a solution containing 50 mM Tris and 25 mM sodium chloride at pH 8.0 and the pass through material was collected. The pass through material was pooled with the load material and filtered through a 0.22 micron membrane. After one passage through the Q Sepharose® column, the endotoxin content was measured by gel clot assay to be between 0.38 to 0.9 EU/mg of anti-M-CSF antibody.

If endotoxin levels are higher than desired at this point (e.g., 3 or higher endotoxin units per milliliter (EU/mL), a second passage through the anion exchange column can optionally be carried out using the procedure outlined above. The anion exchange step can be repeated as many times as desired. Specifically, for a second passage, the column was first regenerated with 3 column volumes of 1.0M sodium chloride in 1M sodium hydroxide, then re-equilibrated with 6 column volumes of a solution containing 50 mM Tris and 25 mM sodium chloride at pH 8.0. Next, the anion exchange eluent material was re-applied to the regenerated and re-equilibrated column. The column was washed again with 4 column volumes of a solution containing 50 mM Tris and 25 mM sodium chloride at pH 8.0 and the pass through material was collected in the flow through fraction. After the second passage through the Q Sepharose® column, the endotoxin levels were reduced to the range of 0.15 to 0.38 EU/mg of anti-M-CSF antibody as measured by gel clot assay.

After the two passes through the Q Sepharose® column, endotoxin levels of the rProtein A eluent material were 0.32 EU/mg anti-M-CSF antibody as determined by the Cambrex Kinetic-Quantitative Chromogenic LAL method.

### Cation Exchange Chromatography

It is at this stage where two different paths can be taken depending on the endotoxin level of the anion exchange eluent material. If the desired endotoxin target level (e.g., less than about 3 endotoxin units per milliliter (EU/mL)) is within about 5 fold of the amount in the anion exchange eluent, the anion exchange eluent can be moved on to the “finishing” step described below.

However, if the anion exchange eluent has endotoxin levels that are higher than desired (e.g., greater than about 3 endotoxin units per milliliter (EU/mL), there is also the potential to add an optional chromatography step comprising a cation exchange column (e.g., SP Sepharose® FF; Amersham, Piscataway, N.J.). The SP Sepharose® column is an ion exchange chromatography column, and in particular, a cation exchange column containing a sulfopropyl group. In order to carry out this step, material from the anion exchange column step was concentrated to about 5-10 mg/ml and dialyzed into an SP Sepharose® equilibration buffer containing 25 mM sodium acetate at pH 5.5. The anion exchange eluent material was then loaded onto the cation exchange column after the resin was equilibrated with a solution containing 25 mM sodium acetate at pH 5.5. The column bed height was 15 cm and the column diameter was between 1 and 5 cm depending upon the amount of the loaded material. The column loading was at about 20 mg protein/ml of resin at a flow rate of 150 cm/hour.

Once the material was adsorbed to the resin, the column was washed with 5 column volumes of a wash solution containing 25 mM sodium acetate (pH 5.5) at a rate of 150 cm/hour. Next, the material was eluted with 8 column volumes of an elution buffer containing 25 mM sodium acetate and 140 mM sodium chloride at pH 5.5. The cation exchange eluent material was then passed on to the finishing step described below.

### Finishing Step

At this point, the eluent material was filtered through a 0.22 micron filter made of polyether sulfone (PES), concentrated (target concentration was greater than 10 mg/ml of 8.10.3F antibody) and then dialyzed for 5 to 8 exchanges (Amicon™ stirred cell concentrator; 30 kDa cut off) into a formulation buffer comprising 20 mM sodium acetate, 140 mM sodium chloride, pH 5.5. After dialyzing the eluent material into the formulation buffer, the endotoxin level was determined to be 0.32 EU/mg as measured by a chromogenic LAL assay.

Once in this buffer, the material was then filtered through a 0.22-micron filter and readied for final polishing using a Millipore Intercept™ Q cartridge (25 mm diameter). Initially, the cartridge was equilibrated with a formulation buffer comprising 20 mM sodium acetate, 140 mM sodium chloride, pH 5.5. Once equilibrated, the material was filtered through two Millipore Intercept™ Q Sepharose®) cartridges in tandem (200 mL max/pair cartridges).

After one passage through the Intercept™ Q cartridge, the endotoxin levels decreased to 0.23 EU/mg anti-M-CSF antibody as measured by the chromogenic LAL method. After one passage, the material was then re-filtered through the cartridges again, and then put through a 0.22-micron filter to yield the final aqueous product comprising anti-M-CSF antibodies 8.10.3F having an amount of endotoxin that was 0.12 EU/mg of anti-M-CSF antibody as measured by the chromogenic LAL assay described in Example 7.

### Example 5

This Example shows a process for reducing the endotoxin content of a clarified broth containing anti-M-CSF 8.10.3F antibodies prepared according to Example 3. The values for the below purifications where a range of endotoxin level is expressed, were all determined using the gel clot assay (See Example 8). Where the endotoxin levels are expressed as a single measurement, the endotoxin level was determined by the Cambrex Kinetic-Quantitative Chromogenic LAL assay (See Example 7).

rProtein A Chromatography

A clarified broth prepared according to Example 3 was loaded directly onto a 150×50 mm column packed with rProtein A Sepharose® FF resin (Amersham, Piscataway, N.J.) equilibrated with an equilibration solution containing 50 mM sodium phosphate and 250 mM sodium chloride at pH 7.0. A resin bed height of 15 cm was used. Loading, washing, and elution for the column used a linear flow rate of 150 cm per hour.

Once the clarified broth was loaded onto the column (maximal load of 25 mg/ml of resin), the column was washed with 5 column volumes of a first wash solution containing 50 mM sodium phosphate (mixture of mono and dibasic sodium phosphate) and 250 mM sodium chloride at pH 7.0, followed by 5 column volumes of a second wash solution containing 25 mM sodium acetate at pH 5.5.

The column was then eluted with 4 column volumes of an elution buffer containing 25 mM sodium acetate at pH 3.5. The eluent was then diluted 1:1 with a solution containing 50 mM Tris and 25 mM sodium chloride at pH 8.0. The pH of the diluted eluent was then adjusted to 8.0 with 1.5 M Tris base and the conductivity was adjusted to be less than 6 mS/cm with sterile water.

### Anion Exchange Chromatography

The rProtein A column eluent was then loaded onto a Q Sepharose® FF column (Amersham, Piscataway, N.J.). The Q Sepharose® column is an ion exchange chromatography column, and in particular, an anion exchange column containing a quaternary ammonium group. As above, a 15 cm bed height was used and the column diameter was varied from 1 to 5 cm (depending on the material load). Before the column was used, it was equilibrated with 6 column volumes of a solution containing 50 mM Tris and 25 mM sodium chloride at pH 8.0. Next, material from the rProtein A column elution was loaded directly onto the anion exchange column at a flow rate of 150 cm/hr. The typical load maximum used for the column was 20 mg/ml of resin. The pass through (non-bound fraction) contained the material of interest, and once all of the material from the rProtein A column was loaded, the column was washed with 4 column volumes of a solution containing 50 mM Tris and 25 mM sodium chloride at pH 8.0 and the pass through material was collected.

The eluent of the rProtein A column was collected and passed through three Q Sepharose® columns. For each additional passage, the anion column was first regenerated with 3 column volumes of 1 M sodium chloride in 1 M sodium hydroxide, then re-equilibrated with 6 column volumes of a solution containing 50 mM Tris and 25 mM sodium chloride at pH 8.0. Next, the anion exchange eluent material was re-applied to the regenerated and re-equilibrated column. The column was washed again with 4 column volumes of a solution containing 50 mM Tris and 25 mM sodium chloride at pH 8.0 and the pass through material was collected in the flow through fraction.

The endotoxin content after the first pass on Q Sepharose® was measured to be between 0.83 to 2.07 EU/mg of anti-M-CSF antibody. After the second pass on Q Sepharose® column, endotoxin levels were measured by gel clot LAL assay to be between 0.52 to 1.03 EU/mg of antibody and after the 3^{rd} pass on Q Sepharose® column the endotoxin was measured to be between 0.29 to 0.58 EU/mg of anti-M-CSF antibody. After the third passage, the final pass through material was pooled with the load material and filtered through a 0.22 micron membrane.

### Cation Exchange Chromatography

Next, the final eluent from the three anion exchange steps were concentrated to about 5-10 mg/ml and dialyzed into an SP Sepharose® equilibration buffer containing 25 mM sodium acetate at pH 5.5. The anion exchange eluent was loaded onto a cation exchange column (e.g., SP Sepharose® FF; Amersham, Piscataway, N.J.) after the resin was equilibrated with an equilibration buffer containing 25 mM sodium acetate at pH 5.5. The SP Sepharose® column is an ion exchange chromatography column, and in particular, a cation exchange column containing a sulfopropyl group. The column bed height was 15 cm and the column diameter was between 1 and 5 cm depending upon the amount of the loaded material. The column loading was at about 20 mg protein/ml of resin at a flow rate of 150 cm/hour.

Once the material was adsorbed to the resin, the column was washed with 5 column volumes of a wash solution containing 25 mM sodium acetate (pH 5.5) at a rate of 150 cm/hour. Next, the material was eluted with 8 column volumes of an elution buffer containing 25 mM sodium acetate and 140 mM sodium chloride at pH 5.5.

After one passage through the SP Sepharose® column, endotoxin content ranged from 0.16 to 1.2 EU/mg of anti-M-CSF antibody. The cation exchange eluent material was then passed on to the finishing step described below.

### Finishing Step

At this point, the eluent material was filtered through a 0.22 micron filter, concentrated (target concentration was greater than 10 mg/ml of 8.10.3F antibody) and then dialyzed for 5 to 8 exchanges (Amicon™ stirred cell concentrator; 30 kDa cut off) into a formulation buffer comprising 20 mM sodium acetate, 140 mM sodium chloride, pH 5.5.

Once in this buffer, the material was then filtered through a 0.22-micron filter and readied for final polishing using a Millipore Intercept™ Q cartridge (25 mm diameter). Initially, the cartridge was equilibrated with a formulation buffer comprising 20 mM sodium acetate, 140 mM sodium chloride, pH 5.5. Once equilibrated, the material was filtered through two Millipore Intercept™ Q Sepharose® cartridges in tandem (200 mL max/pair cartridges).

After one passage through a Q Intercept™ cartridge, endotoxin levels were at 0.16 to 0.32 EU/mg of anti-M-CSF antibody. After one passage, the material was then re-filtered through the cartridges again, and then put through a 0.22-micron filter to yield the final aqueous product comprising anti-M-CSF antibodies 8.10.3F having an amount of endotoxin that was 0.046 EU/mg of antibody as measured by the chromogenic LAL assay described in Example 7.

### Example 6

This Example shows a process for reducing the endotoxin content of a clarified broth containing anti-M-CSF 8.10.3F antibodies prepared according to Example 3. The values for the below purifications where a range of endotoxin level is expressed, were all determined using the gel clot assay (See Example 8). Where the endotoxin levels are expressed as a single measurement, the endotoxin level was determined by the Cambrex Kinetic-Quantitative Chromogenic LAL assay (See Example 7).

rProtein A Chromatography

A clarified broth prepared according to Example 3 was loaded directly onto a 150×50 mm column packed with rProtein A Sepharose® FF resin (Amersham, Piscataway, N.J.) equilibrated with an equilibration solution containing 50 mM sodium phosphate and 250 mM sodium chloride at pH 7.0. A resin bed height of 15 cm was used. Loading, washing, and elution for the column used a linear flow rate of 150 cm per hour.

Once the clarified broth was loaded onto the column (maximal load of 25 mg/ml of resin), the column was washed with 5 column volumes of a first wash solution containing 50 mM sodium phosphate (mixture of mono and dibasic sodium phosphate) and 250 mM sodium chloride at pH 7.0, followed by 5 column volumes of a second wash solution containing 25 mM sodium acetate at pH 5.5.

The column was then eluted with 4 column volumes of an elution buffer containing 25 mM sodium acetate at pH 3.5. The eluent was then diluted 1:1 with a solution containing 50 mM Tris and 25 mM sodium chloride at pH 8.0. The pH of the diluted eluent was then adjusted to 8.0 with 1.5 M Tris base and the conductivity was adjusted to be less than 6 mS/cm with sterile water.

The rProtein A column eluent was measured by gel clot LAL assay to have endotoxin levels between 6.8 to 27.1 EU/mg of anti-M-CSF antibody.

### Anion Exchange Chromatography

The rProtein A column eluent was then loaded onto a Q Sepharose® FF column (Amersham, Piscataway, N.J.). The Q Sepharose® column is an ion exchange chromatography column, and in particular, an anion exchange column containing a quaternary ammonium group. As above, a 15 cm bed height was used and the column diameter was varied from 1 to 5 cm (depending on the material load). Before the column was used, it was equilibrated with 6 column volumes of a solution containing 50 mM Tris and 25 mM sodium chloride at pH 8.0. Next, material from the rProtein A column elution was loaded directly onto the anion exchange column at a flow rate of 150 cm/hr. The typical load maximum used for the column was 20 mg/ml of resin. The pass through (non-bound fraction) contained the material of interest, and once all of the material from the rProtein A column was loaded, the column was washed with 4 column volumes of a solution containing 50 mM Tris and 25 mM sodium chloride at pH 8.0 and the pass through material was collected.

The eluent of the rProtein A column was collected and passed through two Q Sepharose® columns. For the second passage, the anion column was first regenerated with 3 column volumes of 1 M sodium chloride in 1M sodium hydroxide, then re-equilibrated with 6 column volumes of a solution containing 50 mM Tris and 25 mM sodium chloride at pH 8.0. Next, the anion exchange eluent material was re-applied to the regenerated and re-equilibrated column. The column was washed again with 4 column volumes of a solution containing 50 mM Tris and 25 mM sodium chloride at pH 8.0 and the pass through material was collected in the flow through fraction.

The endotoxin content after the first pass on Q Sepharose® was measured to be between 8 to 3.6 EU/mg of anti-M-CSF antibody. After the second passage, the final pass through material was pooled with the load material and filtered through a 0.22 micron membrane.

### Cation Exchange Chromatography

Next, the final eluent from the two anion exchange steps were concentrated to about 5-10 mg/ml and dialyzed into an SP Sepharose® equilibration buffer containing 25 mM sodium acetate at pH 5.5. After concentration and dialysis, the endotoxin levels were measured to be between 1.04 to 2.08 EU/mg of anti-M-CSF antibody.

The anion exchange eluent was loaded onto a cation exchange column (e.g., SP Sepharose® FF; Amersham, Piscataway, N.J.) after the resin was equilibrated with an equilibration buffer containing 25 mM sodium acetate at pH 5.5. The SP Sepharose® column is an ion exchange chromatography column, and in particular, a cation exchange column containing a sulfopropyl group. The column bed height was 15 cm and the column diameter was between 1 and 5 cm depending upon the amount of the loaded material. The column loading was at about 20 mg protein/ml of resin at a flow rate of 150 cm/hour.

Once the material was adsorbed to the resin, the column was washed with 5 column volumes of a wash solution containing 25 mM sodium acetate (pH 5.5) at a rate of 150 cm/hour. Next, the material was eluted with 8 column volumes of an elution buffer containing 25 mM sodium acetate and 140 mM sodium chloride at pH 5.5.

After one passage through the SP Sepharose® column, endotoxin content range from 0.1 to 0.2 EU/mg of anti-M-CSF antibody. The cation exchange eluent material was then passed on to the finishing step described below.

### Finishing Step

At this point, the eluent material was filtered through a 0.22 micron filter, concentrated (target concentration was greater than 10 mg/ml of 8.10.3F antibody) and then dialyzed for 5 to 8 exchanges (Amicon™ stirred cell concentrator; 30 kDa cut off) into a formulation buffer comprising 20 mM sodium acetate, 140 mM sodium chloride, pH 5.5.

Once in this buffer, the material was then filtered through a 0.22-micron filter and readied for final polishing using a Millipore Intercept™ Q cartridge (25 mm diameter. Initially, the cartridge was equilibrated with a formulation buffer comprising 20 mM sodium acetate, 140 mM sodium chloride, pH 5.5. Once equilibrated, the material was filtered through two Millipore Intercept Q cartridges in tandem (200 mL max/pair cartridges).

After one passage through a Q Intercept™ cartridge, endotoxin levels were at 0.16 to 0.32 EU/mg of anti-M-CSF antibody. After one passage, the material was then re-filtered through the cartridges again, and then put through a 0.22-micron filter to yield the final aqueous product comprising anti-M-CSF antibodies 8.10.3F having an amount of endotoxin that was 0.085 EU/mg of anti-M-CSF antibody as measured by the chromogenic LAL assay described in Example 7.

### Example 7

This Example shows a chromogenic LAL assay that was used to determine the amount of endotoxin in the anti-M-CSF antibody compositions in Examples 4 through 6.

Bacterial endotoxin was quantified using the Cambrex Kinetic-Quantitative Chromogenic LAL method (K-QCL) as outlined below. The presence of endotoxin activates the LAL pro-enzyme which catalyzes the splitting of yellow para-nitroaniline (pNA) from a colorless substrate. This colored byproduct was quantitated photometrically at 405 nm. The Reaction Time (time required for appearance of yellow color) is inversely proportional to the amount of endotoxin present. These assays were carried out in 96-well format using an ELISA microplate reader.

First, all work surfaces were wiped down with alcohol or an approved disinfectant. Only pyrogen free reagents, water, and disposables were used. An E. coli endotoxin standard was rehydrated to the EC6 level in the original vial (available as E. coli 055:B5; product number #E50-643; from Cambrex, East Rutherford, N.J.). The reconstitution volume was listed on the Certificate of Analysis. The endotoxin vial was vortexed vigorously for greater than or equal to 10 minutes at 15-30° C. prior to use. The E. coli endotoxin standard vial was labeled with the rehydration time and date, and stored at 2-7° C. The endotoxin standard was available for reuse if within 24 hours. If reused, the endotoxin standard was first incubated at 15-30° C. (≧30 minutes) and vigorously vortexed for greater than or equal to 10 minutes prior to use. A series of dilutions for the endotoxin standard were prepared at the following concentrations (0.005, 0.05, 0.5, 5.0, 50 EU/mL).

Positive controls of all dilutions were prepared in a 96-well ELISA microtiter plate to be examined (10 fold dilution of sample spiked with endotoxin so nominal endotoxin value is 0.5 EU/ml) in order to ensure product inhibition was not occurring.

A series of sample dilutions of the material of interest to be assayed were also prepared in the same 96-well microtiter plate. Up to 1000 fold dilution of the sample was set up in order to prepare pure sample dilutions on the order of ½, ¼, ⅛, and 1/16. An appropriate amount of sample, standard, and water were pipetted into the microtiter plates so that final volumes were consistent (final volume—100 microliters). The microtiter plates were equilibrated for at least 10 minutes at 37° C. 100 microliters of the LAL substrate reagent (Cambrex, #K50-643, or equivalent) that has been reconstituted in 50 mM Tris Buffer (Cambrex #S50-642, or equivalent; each vial reconstituted with 2.6 mL of Tris buffer) was added to the microtiter plate. Once the LAL substrate is added, the microtiter plate was placed in an ELISE plate reader (Bio-Tek ELx808, or equivalent, equipped with a 405 nm filter) and a reading cycle was initiated. The ELISA reader was driven by Cambrex Kinetic-QCL Software, (Version 2.0). Once the assay was finished, the software was used to calculate the endotoxin concentration in the sample. The following equation was used to calculate EU/mg A₂₈₀ of antibody. The equation is the known endotoxin concentration (in EU/mL) divided by the protein concentration (in mg/mL), and the result equals the concentration in EU/mg A₂₈₀.

### Example 8

This Example shows a gel clot LAL assay that was used to determine the amount of endotoxin in the anti-M-CSF antibody compositions in Examples 4 through 6.

A series of dilutions of the sample material to be assayed were prepared in a Biohazard hood that had been wiped down with ethanol and allowed to run for 15 minutes prior to initiation of activities. The sample material was diluted 10-fold and 20-fold in 10×75 mm pyrogen-free glass tubes using sterile water for injection. At the higher dilutions, pyrosol (Cape Cod Associates, #BR051) was added with a pH indicator to ensure the higher amount of sample material does not change the pH of the test solution.

Positive controls were set up in parallel at the selected dilutions to ensure product inhibition was not occurring. In general, a final target volume of 1 milliliter for each dilution was achieved. Once the dilutions were made, 0.2 milliliter of each dilution was pipetted into single test tubes containing the clotting agent (Cape Cod Associates, #GS006 for 0.06 EU/ml test). The tubes were stoppered and then incubated at 37° C. for 60 minutes.

The tubes were then turned over. If the clot remained at the bottom of the tube, it was considered positive for the presence of endotoxin. If liquid ran down the tube, it was considered negative. Based on the dilution used, endotoxin levels were then calculated within a particular range and reported in Examples 4 through 6. For example, if a 10-fold dilution was positive and a 20-fold dilution was negative in a 0.06 EU/ml test kit, the endotoxin levels in the sample were considered to be between 0.6 and 1.2 EU/ml. Following the equation for conversion described in Example 7, the EU/mg A₂₈₀ of antibody was determined.

### Example 9

This example illustrates the production of a liquid pharmaceutical composition containing anti-M-CSF antibody 8.10.3F, L-histidine monohydrochloride monohydrate, disodium ethylenediaminetetraacetic acid dihydrate, mannitol, and polysorbate 80.

TABLE 3
Description of anti-M-CSF antibody 8.10.3F formulation.

| | | | Antibody |
|---|---|---|---|
| | | | concen- |
| | | | tration, |
| Description | pH | Appearance | (mg/ml) |
| 10 mM histidine, 45 mg/ml mannitol, | 6.0 | Clear and | 8.4 |
| 0.02 mg/ml disodium EDTA dihydrate, | | colorless | |
| 0.2 mg/ml polysorbate 80 | | | |

### Preparation of the Formulation

Materials which were used in preparation of the formulations include: mannitol, histidine, polysorbate 80, disodium ethylenediaminetetraacetic acid dihydrate, water for injection, hydrochloric acid/sodium hydroxide, which were used as dilute solutions to adjust the pH, and an antibody stock solution (e.g., monoclonal anti-M-CSF antibody 8.10.3F purified material prepared according to Example 4, but dialyzed into a histidine, EDTA and mannitol formulation solution).

Formulation solution ingredients were as follows: 45 grams per liter (g/L) of mannitol, 1.55 g/L of histidine, 0.02 g/L of disodium ethylenediaminetetraacetic acid dihydrate. A 1 M hydrochloric acid solution was prepared by appropriate dilution from concentrated hydrochloric acid with water for injection. A solution was then prepared by dissolving the preceding ingredients (as described above) in water at about 90% of the desired volume: mannitol, histidine, disodium ethylenediaminetetraacetic acid dihydrate. After addition of all of the excipients except polysorbate 80, dissolution was achieved, and the pH of the solution was adjusted to pH 6 with a 1M hydrochloric acid solution. After the addition of the hydrochloric acid solution, the final quantity of the water was added to bring the volume up to 100% of the desired amount. The buffer solution was then filtered (0.22 micron filter) into a sterilized receptacle.

A 20 g/L polysorbate 80 solution was prepared by appropriate dilution of a 100% polysorbate 80 solution by the above-prepared formulation buffer (45 g/L of mannitol, 1.55 g/L of histidine, 0.02 g/L of disodium ethylenediaminetetraacetic acid dihydrate, pH 6).

The anti-M-CSF antibody material underwent a buffer exchange for three cycles as follows. The antibody solution was diluted ten times with the desired buffer solution and centrifuged at 3000×g with a molecular weight cut-off membrane (e.g., 30 kD) to reduce the volume ten fold. This cycle was repeated for a total of three times. The final volume of the antibody solution was adjusted by appropriate dilution to achieve the desired antibody concentration of 8.4 mg/ml. An addition of 20 g/L polysorbate 80 solution was made to achieve 0.2 g/L polysorbate 80 in the antibody formulation.

The formulations were then filtered through 0.2μ sterilizing grade filters and filled into vials. A fill-volume of 0.5 to 1 ml was used in 2 ml Type 1 glass vials. The vials were closed with Dalkyo 777-1 Fluorotec® coated stoppers and crimp sealed.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

As various changes could be made in the above methods and compositions without departing from the scope of the invention, it is intended that all matter contained in the above description shall be interpreted as illustrative and not in a limiting sense. In addition, it should be understood that aspects of the various embodiments may be interchanged both in whole or in part.

### SEQUENCES

SEQ ID NO: 1

atggagttggggctgtgctgggttttccttgttgctattttagaaggtgt
ccagtgtgaggtgcagctggtggagtctgggggaggcttggtacagcctg
gggggtccctgagactctcctgtgcagcctctggattcaccttcagtagt
tttagtatgacctgggtccgccaggctccaggaaaggggctggagtgggt
ttcatacattagtagtagaagtagtaccatatcctacgcagactctgtga
agggccgattcaccatctccagagacaatgccaagaactcactgtatctg
caaatgaacagcctgagagacgaggacacggctgtgtattactgtgcgag
agatcctcttctagcgggagctaccttctttgactactggggccagggaa
ccctggtcaccgtctcctcagcctccaccaagggcccatcggtcttcccc
ctggcgccctgctccaggagcacctccgagagcacagcggccctgggctg
cctggtcaaggactacttccccgaaccggtgacggtgtcgtggaactcag
gcgctctgaccagcggcgtgcacaccttcccagctgtcctacagtcctca
ggactctactccctcagcagcgtggtgaccgtgccctccagcaacttcgg
cacccagacctacacctgcaacgtagatcacaagcccagcaacaccaagg
tggacaagacagttgagcgcaaatgttgtgtcgagtgcccaccgtgccca
gcaccacctgtggcaggaccgtcagtcttcctcttccccccaaaacccaa
ggacaccctcatgatctcccggacccctgaggtcacgtgcgtggtggtgg
acgtgagccacgaagaccccgaggtccagttcaactggtacgtggacggc
gtggaggtgcataatgccaagacaaagccacgggaggagcagttcaacag
cacgttccgtgtggtcagcgtcctcaccgttgtgcaccaggactggctga
acggcaaggagtacaagtgcaaggtctccaacaaaggcctcccagccccc
atcgagaaaaccatctccaaaaccaaagggcagccccgagaaccacaggt
gtacaccctgcccccatcccgggaggagatgaccaagaaccaggtcagcc
tgacctgcctggtcaaaggcttctaccccagcgacatcgccgtggagtgg
gagagcaatgggcagccggagaacaactacaagaccacacctcccatgct
ggactccgacggctccttcttcctctacagcaagctcaccgtggacaaga
gcaggtggcagcaggggaacgtcttctcatgctccgtgatgcatgaggct
ctgcacaaccactacacgcagaagagcctctccctgtctccgggtaaa

SEQ ID NO: 2

MELGLCWVFLVAILEGVQCEVQLVESGGGLVQPGGSLRLSCAASGFTFSS
FSMTWVRQAPGKGLEWVSYISSRSSTISYADSVKGRFTISRDNAKNSLYL
QMNSLRDEDTAVYYCARDPLLAGATFFDYWGQGTLVTVSSASTKGPSVFP
LAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCP
APPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPAP
IEKTISKTKGQPREPQVYTLPPSREEMTKNQVSLTCLVKGFYPSDIAVEW
ESNGQPENNYKTTPPMLDSDGSFFLYSKLTVDKSRWQQGNVFSCSVMHEA
LHNHYTQKSLSLSPGK

SEQ ID NO: 3

atggaaaccccagcgcagcttctcttcctcctgctactctggctcccaga
taccaccggagaatttgtgttgacgcagtctccaggcaccctgtctttgt
ctccaggggaaagagccaccctctcctgcagggccagtcagagtgttagc
agcagttacttagcctggtaccagcagaaacctggccaggctcccaggct
cctcatctatggtgcatccagcagggccactggcatcccagacaggttca
gtggcagtgggtctgggacagacttcactctcaccatcagcagactggag
cctgaagattttgcagtgtattactgtcagcagtatggtagctcacctct
cactttcggcggagggaccaaggtggagatcaaacgaactgtggctgcac
catctgtcttcatcttcccgccatctgatgagcagttgaaatctggaact
gcctctgttgtgtgcctgctgaataacttctatcccagagaggccaaagt
acagtggaaggtggataacgccctccaatcgggtaactcccaggagagtg
tcacagagcaggacagcaaggacagcacctacagcctcagcagcaccctg
acgctgagcaaagcagactacgagaaacacaaagtctacgcctgcgaagt
cacccatcagggcctgagctcgcccgtcacaaagagcttcaacaggggag
agtgt

SEQ ID NO: 4

METPAQLLFLLLLWLPDTTGEFVLTQSPGTLSLSPGERATLSCRASQSVS
SSYLAWYQQKPGQAPRLLIYGASSRATGIPDRFSGSGSGTDFTLTISRLE
PEDFAVYYCQQYGSSPLTFGGGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC

What is claimed is:

1. A composition comprising:
at least one antibody comprising:
an amino acid sequence that is at least 90% identical to a light chain amino acid sequence shown in SEQ ID NO: 4; and
an amino acid sequence that is at least 90% identical to a heavy chain amino acid sequence shown in SEQ ID NO: 2,
wherein the antibody binds to human M-CSF and the composition is substantially free of endotoxin.

2. The composition according to claim 1, wherein the composition is a liquid composition, the antibody is a human IgG2 antibody, and the antibody does not comprise a signal sequence.

3. The composition according to claim 2, wherein the antibody comprises a heavy chain amino acid sequence with at least 99% sequence identity to SEQ ID NO: 2 and a light chain amino acid sequence with at least 99% sequence identity to SEQ ID NO: 4.

4. The composition according to claim 2, wherein the antibody comprises a heavy chain amino acid sequence comprising the variable region of SEQ ID NO: 2 and a light chain amino acid sequence comprising the variable region of SEQ ID NO: 4.

5. The composition according to claim 3, wherein the antibody comprises a heavy chain amino acid sequence comprising SEQ ID NO: 2 and a light chain amino acid sequence comprising SEQ ID NO: 4.

6. The composition according to claim 1, wherein the antibody comprises an isolated human monoclonal IgG2 anti-M-CSF antibody having the heavy and light chain amino acid sequences of antibody 8.10.3F.

7. The composition according to claim 1, wherein the composition has a concentration of endotoxin of from about 0.001 to about 1.6 endotoxin units per milligram of antibody (EU/mg).

8. The composition according to claim 1, wherein the composition has a concentration of endotoxin of from about 0.5 endotoxin units per milliliter (EU/mL) to about 3.0 endotoxin units per milliliter (EU/mL).

9. The composition according to claim 1, wherein the presence of endotoxin is determined by a chromogenic LAL assay.

10. A method of purifying a monoclonal IgG antibody comprising:
contacting the antibody with an affinity chromatography resin that binds to the antibody;
washing the affinity chromatography resin with a wash solution comprising phosphate ions and chloride ions;
washing the affinity chromatography resin with a wash solution comprising acetate ions at pH 5.5;
eluting the antibody from the affinity chromatography resin to form an affinity chromatography eluent comprising the antibody;
contacting the affinity chromatography eluent with an ion-exchange resin that binds to the antibody; and
eluting the antibody from the ion-exchange resin.

11. A method of reducing the amount of endotoxin in a composition comprising at least one antibody comprising an amino acid sequence that is at least 90% identical to a light chain amino acid sequence shown in SEQ ID NO: 4, and further comprising an amino acid sequence that is at least 90% identical to a heavy chain amino acid sequence shown in SEQ ID NO: 2, wherein the antibody binds to human M-CSF, the method comprising:
contacting the composition with an affinity chromatography resin that binds to the antibody;
eluting the antibody from the affinity chromatography resin to form an affinity chromatography eluent comprising the antibody;
contacting the affinity chromatography eluent with an ion-exchange resin that binds to the antibody; and
eluting the antibody from the ion-exchange resin, wherein the antibody is substantially free of endotoxin.

12. A liquid pharmaceutical composition comprising:
a pharmaceutically acceptable excipient; and
at least one antibody comprising:
an amino acid sequence that is at least 90% identical to a light chain amino acid sequence shown in SEQ ID NO: 4,
an amino acid sequence that is at least 90% identical to a heavy chain amino acid sequence shown in SEQ ID NO: 2,
wherein the antibody binds to human M-CSF and the composition is substantially free of endotoxin.

13. A method for the treatment of a M-CSF-mediated disorder in a subject, comprising administering to the subject a therapeutically effective amount of a liquid pharmaceutical composition comprising: at least one antibody comprising an amino acid sequence that is at least 90% identical to a light chain amino acid sequence shown in SEQ ID NO: 4, and further comprising an amino acid sequence that is at least 90% identical to a heavy chain amino acid sequence shown in SEQ ID NO: 2, wherein the antibody binds to human M-CSF and the composition is substantially free of endotoxin; and a pharmaceutically acceptable excipient.

14. The method according to claim 13, wherein the M-CSF-mediated disorder is a neoplasia disorder.

15. The method according to claim 13, wherein the M-CSF-mediated disorder is an inflammatory disease.
